(19) United States
(12) Patent Application Publication
(10) Pub. No.: US 2026/0348967 A1
(43) Pub. Date: Nov. 5, 2026

(54) CD207 Dendritic Cells as a Biomarker for Tumoural Progression and Therapeutic Target

(71) Applicants: Institut Gustave-Roussy, Villejuif (FR); INSTITUT NATIONAL DE LA SANTE ET DE LA RECHERCHE MEDICALE, Paris (FR); UNIVERSITE PARIS-SACLAY, GIF-SUR-YVETTE (FR)

(72) Inventors: Florent GINHOUX, Paris (FR); Charles-Antoine DUTERTRE, Malakoff (FR)

(73) Assignees: Institut Gustave-Roussy, Villejuif (FR); INSTITUT NATIONAL DE LA SANTE ET DE LA RECHERCHE MEDICALE, Paris (FR); UNIVERSITE PARIS-SACLAY, GIF-SUR-YVETTE (FR)

(21) Appl. No.: 19/119,785

(22) PCT Filed: Oct. 17, 2023
(86) PCT No.: PCT/EP2023/078773

§ 371 Date: Apr. 10, 2025

(30) Foreign Application Priority Data
Oct. 17, 2022 (EP) 22306572.3

(51) Int. Cl. G01N 33/575; C07K 16/28; C12Q 1/6886

(52) U.S. Cl. CPC G01N 33/5759; C07K 16/2851; C12Q 1/6886; C12Q 2600/118; C12Q 2600/158; G01N 2333/70539; G01N 2333/70596

(57) ABSTRACT

The present invention proposes an efficient method to prognose the evolution of a solid cancer in a subject in need thereof, by analysing the frequency of a particular subset of dendritic cells as well as their relative number as compared to that of another subset of dendritic cells in a tumour sample of said subject. More precisely, the method of the invention comprises the step of detecting and quantifying the frequency of CD207⁺ conventional dendritic cells of type 2 and 3 (“CD207⁺ DC2+DC3”) in a tumour sample of said subject. If the frequency of said cells is high, in particular in the tumour core, then it means that the subject has a bad prognosis. The method of the invention also comprises the calculation within the tumour of the ratio of the number of conventional dendritic cells of type 1 (cDC1) to the number of CD207⁺ DC2+DC3 to predict T-cell clonality in the tumour. As a matter of fact, the inventors herein establish that the CD207⁺ DC2+DC3 participate in the regulation of the anti-tumour T-cell cytotoxicity by limiting T-cell clonality and impairing the CD8⁺ tissue resident memory T cell (TRM) response. Their analysis furthermore reveals that the CD207⁺ DC2+DC3 are enriched in biopsies from later deceased patients as compared to alive patients suffering from different solid cancers. The inventors also propose to reduce the abundance of these cells within the tumour core, e.g., by appropriate drugs, in order to limit the development of said solid tumour in the subject.

### SUMMARY

The present invention proposes an efficient method to prognose the evolution of a solid cancer in a subject in need thereof, by analysing the frequency of a particular subset of dendritic cells as well as their relative number as compared to that of another subset of dendritic cells in a tumour sample of said subject. More precisely, the method of the invention comprises the step of detecting and quantifying the frequency of CD207⁺ conventional dendritic cells of type 2 and 3 (“CD207⁺ DC2+DC3”) in a tumour sample of said subject. If the frequency of said cells is high, in particular in the tumour core, then it means that the subject has a bad prognosis. The method of the invention also comprises the calculation within the tumour of the ratio of the number of conventional dendritic cells of type 1 (cDC1) to the number of CD207⁺ DC2+DC3 to predict T-cell clonality in the tumour. As a matter of fact, the inventors herein establish that the CD207⁺ DC2+DC3 participate in the regulation of the anti-tumour T-cell cytotoxicity by limiting T-cell clonality and impairing the CD8⁺ tissue resident memory T cell (T_{RM}) response. Their analysis furthermore reveals that the CD207⁺ DC2+DC3 are enriched in biopsies from later deceased patients as compared to alive patients suffering from different solid cancers. The inventors also propose to reduce the abundance of these cells within the tumour core, e.g., by appropriate drugs, in order to limit the development of said solid tumour in the subject.

### DESCRIPTION OF THE PRIOR ART

The mononuclear phagocyte system (MPS) was initially comprised of monocytes and macrophages, which later included conventional dendritic cells (cDCs) discovered by Ralph Steinman and colleagues (Steinman and Cohn, 1973). These cells were shown to be the unique potent activators of naïve T-cells and were viewed as ‘accessory’ cells bridging the innate and adaptive immune response (Nussenzweig et al., 1980; Steinman and Witmer, 1978; Steinman et al., 1983). The identification of common DC progenitors (CDPs) helped in defining the cDC lineage and the ontogeny of various DC subsets (Lee et al., 2015; Liu et al., 2009; Naik et al., 2007; Onai et al., 2007), identifying three major DC subsets, namely, cDC1s, CD1c⁺ DCs (initially called cDC2) and plasmacytoid DCs (pDCs) across tissues and species (Guilliams et al., 2016). However, with the era of high dimensional single-cell technologies, further subsets and states have been described notably within the human CD1c⁺ and pDC compartments (Bourdely et al., 2020; Cytlak et al., 2019; Dutertre et al., 2019; See et al., 2017; Villani et al., 2017). Additionally, CD123⁺ DCs were shown to harbour both bona fide pDCs and DC precursors (pre-DCs) (See et al., 2017; Villani et al., 2017). CD1c⁺ “cDC2s” were shown to comprise CD5⁺ cDC2s and a subset of CD5 CD14^{+/-} DC3, the latter of which has been shown to expand during inflammation (Bourdely et al., 2020; Dutertre et al., 2019). Whether DC3s belong formally to the cDC lineage through a shared development from the common DC progenitor (CDP)/pre-DCs still remains an open and important question. Indeed, recent studies showed that DC3s could derive directly from a GMP/MDP progenitor, independently of CDPs/pre-DCs (Bourdely et al., 2020; Cytlak et al., 2019). The DC nomenclature is driven by their ontogeny and it was established that the term “conventional” DC (CDC) would be used to qualify CDP/pre-DC-derived DCs (Guilliams et al., 2014). Thus, the common myeloid progenitor origin of pre-DC-derived cDC1 and DC2, as well as GMP/MDP-derived DC3, allows to classify these three cell subsets as myeloid DCs (mDCs).

While cell types/subsets are determined by their ontogeny, cell states are molecular programmes that could be acquired in a cell's lifespan in response to specific tissue or inflammatory cues (Ginhoux et al., 2022). One example is the mature DCs enriched in immunoregulatory molecules (mregDCs), which have been recognized as terminally differentiated and matured states of cDC1s, DC2s and potentially DC3s, and are implicated in tumour immunity (Di Pilato et al., 2021; Maier et al., 2020; Zheng et al., 2017).

The increased accessibility and feasibility of single-cell RNA sequencing (scRNAseq) is a double-edged sword. Whilst the public database continues to expand, the art is overwhelmed with numerous publications with different annotations and names of DC clusters, resulting in misalignment of the nomenclature of cell populations (Ginhoux et al., 2022). For example, ‘DC3’ was initially coined to describe a subset of inflammatory CD5 CD14^{+/-} CD1c⁺ mDCs (Bourdely et al., 2020; Cytlak et al., 2019; Dutertre et al., 2019), whereas others have used this term to describe cells (Di Pilato et al., 2021; Gerhard et al., 2020; Zilionis et al., 2019) that have an obvious transcriptomic alignment to mregDCs (Maier et al., 2020). Furthermore, a CD207⁺ (Langerin) population has been described in various contexts (Bell et al., 1999; Leader et al., 2021; Zhang et al., 2021). Evidently, there is little consensus on how these subsets are characterised, which confuses both experts and new researchers entering the field (Ginhoux et al., 2022). In this context, there is a need to clarify and ascertain which subtype of dendritic cells is involved in the pathogenicity of solid cancers.

Currently, the prognostic of solid cancers is often evaluated by analysing the localisation and the clonality of tumour-infiltrated lymphocytes (TILs) {Tsuji et al., 2020; Valpione et al., Nat Comm 2021}. This clonality is generally assessed by means of single-cell TcR-sequencing, which is the only way to detect their degree of overlap with peripheral repertoire, and the presence of detectable spontaneous anti-tumour immune response in the patients. Yet, single-cell TcR-sequencing is very expensive and time-consuming and can therefore not be used routinely. There is therefore a need of an easiest and more economical way to evaluate the prognostic of a patient, in response to a therapy or before the therapy has started.

### DETAILED DESCRIPTION OF THE INVENTION

Recently, monocytes and macrophages from 41 scRNAseq datasets across various human tissues in health and disease were integrated to unify the identities of these cells (Mulder et al., 2021). In a first part, the results exposed below adopted a similar approach to obtain a comprehensive and unbiased DC atlas that will help in realigning their nomenclature. The mDC-VERSE allowed to provide an in-depth analysis of DC2 and DC3 heterogeneity and understand the potential significance of these cell types in cancer settings.

In a second part, the inventors studied the localisation and the functionality of a particular subset of dendritic cells, herein interchangeably called “CD207 DC2+DC3s” or “CD207⁺ DC2+DC3s” in various solid tumours. Spatial transcriptomics from human breast cancers have shown in the past that these cells localised within the tumour stromal region, contrary to most other immune cells. This observation complemented previous findings, where these cells had a high number of interactions with fibroblasts (Kim et al., 2020). However, the present data (FIG. 2) rather show that CD207⁺ DC2+DC3s in fact specifically accumulate within the tumour core, more precisely within the tumour glandular areas of solid tumour, surprisingly far away from the B and T lymphocytes that rather accumulate in the tumour stroma. The inventors also demonstrate that these particular cells are mainly enriched in patients without T-cell clonal expansion, contrary to mregDCs and cDC1s that behave oppositely. Notably, the inventors herein show that a common state of DC2+DC3s that co-expressed CD207 and CD1a (CD207⁺ DC2+DC3) (1) accumulate inside the tumour of most cancer studied; (2) inversely correlate with T-cell clonal expansion, and with the frequency of CD8⁺ resident memory T-cells (T_{RMs}, that are classically defined as co-expressing CD69 and CD103); (3) are mostly detected embedded within tumour nests while all the other DCs, T and B cells were mainly detected within the tumour stroma of breast and lung adenocarcinomas.

The present inventors more importantly show (FIG. 4) that there is a negative correlation of CD207 DC2+DC3s with resident memory CD8⁺ T-cells (hereafter “CD8⁺ T_{RM}-cells” that are classically defined as co-expressing CD69 and CD103) which are known to accumulate in various cancers and are associated with improved disease outcomes and survival (Park et al., 2019). The presence of CD207 DC2+DC3s on the contrary strongly positively correlated with terminally differentiated effector memory T-cells that re-express CD45RA (hereafter “CD8⁺ T_{EMRA} cells”), which have been shown to be senescent/hypofunctional cells (Reading et al., 2018). Altogether, these observations allowed the inventors to hypothesize that CD207 DC2+DC3s could participate in the regulation of the anti-tumour T-cell cytotoxicity, for example by limiting T-cell clonality in the tumour core. They therefore propose to use the CD207 DC2+DC3s signature to predict T-cell clonality within solid tumours.

High T-cell clonality being a sign of good prognostic for a patient suffering from a solid tumour, the inventors finally analysed the Disease-Specific Survival (DSS) of patients, depending on the presence or not of high levels of CD207 DC2+DC3 in their tumour core. They observed that, surprisingly, high levels of CD207 DC2+DC3 in tumour nests associate with cancer-related deaths in all the seven TCGA cancer datasets analysed as compared to alive patients (FIG. 3F) and in other independent cohorts of patients (FIG. 3G).

In particular, the inventors demonstrate that CD207 DC2+DC3s could also serve as a prognostic factor by calculating the ratio between the number of CD207 DC2+DC3s with respect to the number of cDC1s within the tumour core, as their results show that this ratio predicts efficiency and reliably whether patients would develop a T-cell clonal expansion (FIG. 3B). The CD207 DC2+DC3s to cDC1s ratio is also proposed as a reliable mean to predict the progression of solid tumours.

Importantly, high expression of CD207 within tumors was never associated with bad prognosis or with the efficiency of an anti-cancer treatment in any prior study. On the contrary, high expression of this protein in tumors or in Langerhans cells has been reported in the past not to be correlated (US 2004/229297) or inversely correlated (Dyduch G. et al, 2017; Zilionis et al. 2019) with patient survival.

### Methods for Prognosing Solid Tumours

The present inventors have surprisingly found that solid tumours display a higher proportion of a specific class of dendritic cells, namely CD207⁺ conventional dendritic cells of type 2 and 3 (DC2+DC3).

More specifically, the present inventors have found that the population of DC2+DC3 dendritic cells expressing the biomarkers CD207 and CD1a (the so-called CD207⁺ DC2+DC3) are hyper-represented in the tumour core of patients suffering from a severe cancer associated with bad prognosis. The proportion of this population of dendritic cells in the tumour core of these patients is much higher than in the stroma of said tumour or in the tumour core of patients suffering from a cancer with good prognosis. As shown on FIGS. 3F, 3G and 3K (which evaluate the predictive impact of the expression of genes specifically expressed by dendritic cell subpopulations (CD207⁺ DC, DC1 and mregDC) on the survival of patients with a broad spectrum of solid tumors who received therapeutic antibodies (anti-PD-1, PD-L1 or CTLA4), the proportion of CD207⁺ DC2+DC3 is sufficient to discriminate between numerous cancer patients having a good versus a bad prognosis. Therefore, the high frequency of CD207⁺ DC2+DC3 in the tumour core can be used as a negative prognosis factor for a number of solid cancers.

The invention thus enables the skilled person to identify those subjects suffering from a solid cancer will have a short survival, simply by quantifying the frequency of CD207⁺ DC2+DC3 in a tumour sample from said subjects. Whereas the method of prior art relied on a costly and time-consuming analysis (single-cell TcR-sequencing), a unique and rapidly obtained parameter is used in the method of the invention. This parameter can be determined in few hours, when flow cytometry is used. When immunohistochemistry is used, it is possible to obtain the prognosis in about two days' time. When RNAseq is used, the timing is longer (at least two weeks) but it is always less than the current 3 months required by the methods involving single-cell TCR-sequencing. Thus, the method of the invention is particularly advantageous because it generates a prognosis in a very short time and with a very high degree of confidence, whereas the method currently recommended by WHO (single-cell TcR-sequencing) is both time-consuming and costly.

In a first aspect, the present invention thus provides an in vitro method to prognose the evolution of a solid cancer in a subject in need thereof or to monitor or follow-up the evolution of a solid cancer, or for determining the outcome of a solid cancer.

Said method comprises the steps of:
a) Detecting and quantifying the quantity, density or frequency of CD207⁺ conventional dendritic cells of type 2 (DC2+DC3) cell population in a tumour sample of said subject,
b) Comparing the value of step a) to a reference value, and
c) Prognosing that the subject has a bad prognosis if the quantity, density or frequency of CD207⁺ DC2+DC3 cell population is higher than said reference value.

The CD207⁺ conventional dendritic cells of type 2 (DC2+DC3) are herein called “dendritic cells of the invention”. These cells are dendritic cells expressing high level of CD1a and CD207 proteins or high level of CD1A and CD207 transcripts (mRNAs).

The sequence of the cluster of differentiation CD1a is well-known. This member of the CD1 family is a transmembrane glycoprotein that is structurally related with the major histocompatibility Complex (MHC) proteins and forms heterodimers with the β-2-microglobuline. In human, the protein has the sequence displayed in NP_001307581 and NP_001754, encoded by the NM_001763 and NM_001320652 mRNAs. Antibodies recognizing CD1a are commercially available. They can be used for ELISA, flow cytometry, IHC, and western-blots.

The CD207 marker is also called Langerin. It is a type II transmembrane protein expressed by Langerhans cells and dendritic cells. Langerin recognizes and binds carbohydrates, such as mannose, fucose and N-acetylglucosamine. It contributes to the binding of the antigen to CD1a molecule. In mice, langerin is involved in antigen binding to MHC II glycoproteins and to MHC I glycoproteins during cross-presentation. In human, the protein NP_056532 is encoded by NM_015717 mRNA. Antibodies recognizing Langerin are commercially available. They can be used for ELISA, flow cytometry, IHC, and western-blots.

In the context of these methods, the term “tumour sample” or “solid cancer sample” means a sample containing a detectable amount of tumour cells. Such solid cancer sample allows the skilled person to perform any type of measurement of the level of the dendritic cells of the invention. In some cases, the methods according to the invention may further comprise a preliminary step of taking a solid cancer sample from the patient. By a “solid cancer sample”, it is referred to a tumour tissue sample. Even in a cancerous patient, the tissue which is the site of the tumour still comprises non tumour healthy tissue. The “cancer sample” should thus be limited to tumour tissue taken from the patient. Said “cancer sample” may be a biopsy sample or a sample taken from a surgical resection therapy. These samples are preferably maintained in appropriate conditions so that they are not altered after their collect from the subject's body. In particular, for flow cytometry, following cell isolation using conventional enzymatic digestion, isolated cells can be maintained in freezing conditions and/or suspended immediately analysed by flow cytometry to evaluate their frequency among CD45⁺ leukocytes. Alternatively, the cancer sample can be embedded in paraffin or snap-frozen followed by analysis of CD207⁺ cells by immunohistochemistry.

In a preferred embodiment, the tissue is a diseased tissue. In a preferred embodiment of the method, the tissue is a tumour or a biopsy thereof. In a preferred embodiment of the method, a tissue or a biopsy thereof is first excised from a patient, and the levels of the cells of the invention in the tissue or biopsy are then determined in an immunoassay with the antibodies or antibody fragments described below.

In a preferred embodiment, step a) of the method of the invention is achieved by flow cytometry by assessing the relative frequency of CD207⁺ DC2+DC3 cells among CD45⁺ leukocytes on a sample of the tumour, preferably on a sample of the tumour core.

Flow cytometry is a powerful technology that allows researchers and clinicians to perform complex cellular analysis quickly and efficiently by analysing several parameters simultaneously. The amount of information obtained from a single sample can be further expanded by using multiple fluorescent reagents. The information gathered by the flow cytometer can be displayed as any combination of parameters chosen by the skilled person. Cells pass single-file through a laser beam. As each cell passes through the laser beam, the cytometer records how the cell or particle scatters incident laser light and emits fluorescence. Using a flow cytometric analysis protocol, one can perform a simultaneous analysis of surface molecules at the single-cell level.

More preferably, the detection of the cell surface antigens in the methods of the invention is performed by an exclusion gating strategy by flow cytometry.

In this preferred embodiment, the step a) of the invention requires to exclude the contaminant cells from the population of dendritic cells type 2 and 3 expressing CD207 and CD1a.

As used herein, “contaminant cells” refer to granulocytes, e.g. neutrophils, eosinophils, basophils, mast cells, monocytes, macrophages and lymphocytes, e.g., T cells, NK cells, ILCs, B cells, but also precursors of these cell types. The existence of markers which are specific for each of the contaminant cell types enables the identification of these cells in the sample of the subject. Identified contaminant cells can then be removed from the sample (i.e., physically) or from the analysis (i.e., by retaining only the data pertaining to the dendritic cell population for the analysis), so that the study then only focuses on the dendritic cell population. In this respect, although any of the above-mentioned analytical techniques can be used to identify the said contaminant cells, flow cytometry is particularly adapted for this task, since it enables the skilled person to eliminate the contaminants and analyse the dendritic cell population with minimal effort. In this respect, any antibodies directed against one or more antigens expressed by one or more of the contaminant cells can be used to identify the said contaminant cells. In a particular embodiment, antibodies specific for well-known antigens expressed by granulocytes (CD15, CD16), T lymphocytes (CD3), B lymphocytes (CD19), and/or NK cells (CD16) can be used in step a). Using anti-CD15, anti-CD16, anti-CD3 or anti-CD19 antibodies therefore enables to detect and therefore exclude the cells expressing CD15, CD16, CD3 and CD19 proteins, notably the CD3⁺ T lymphocytes, the CD16⁺ NK cells, as well as the CD15⁺ or CD16⁺ granulocytes. In a preferred embodiment, the antibodies used to identify and/or to remove the contaminant cells according to the method of the invention comprise anti-CD15, anti-CD16, anti-CD3, and anti-CD19 antibodies. Of note, anti-CD15 antibodies may be used instead of anti-CD16 antibodies to detect the granulocytes.

Cells expressing CD45 at their surface are all human leucocytes (more precisely, lymphocytes, eosinophils, monocytes, basophils and neutrophils, and dendritic cells with different level of expression). This cluster of differentiation is however absent from erythrocytes and platelets. In a particular embodiment, it is advantageous to analyse only the CD45 expressing-cells, in order to eliminate the contaminant blasts and to select mature cells, including all the dendritic cells and monocytes. In this embodiment, the dendritic cells are detected in the CD45⁺ population of the cells present in the tumour sample. After exclusion of other contaminating populations, the expression level of CD1a and/or CD207 can be assessed (see FIG. 6).

Thus, in a preferred embodiment when flow cytometry is used for step a), the first step of the method of the invention comprises the detection and the measurement of CD45 expression at the cell surface of the cells present in the tumour sample.

The sequence of the cluster of differentiation CD45 is well-known. The CD45 molecules are single chain integral membrane proteins, comprising at least 5 isoforms, ranging from 180 to 220 kDa. They are generated by alternative splicing combinations of three exons (A, B, and C) of the genomic sequence. The non-restricted CD45 antigen, Leucocyte Common Antigen (LCA) consists of an extracellular sequence, proximal to the membrane, which is common to all CD45 isoforms. All the monoclonal antibodies that belong to the CD45 cluster react with this part of the antigen and are able to recognize all CD45 isoforms. These isoforms have extra-cytoplasmic sequences ranging from 391 to 552 amino acids long, with numerous N-linked carbohydrate attachment sites. The cytoplasmic portion contains two phospho-tyrosine-phosphatase domains.

In a preferred embodiment, the DC2+DC3 cells of the invention are more precisely identified by flow cytometry as proposed on FIG. 6, i.e., by selecting live cells, then CD45⁺ cells, then, among these CD45⁺ cells, CD3⁻CD16⁻CD19 CD20⁻ cells that are also HLA-DR⁺, then, among these CD3⁻CD16⁻CD19⁻CD20⁻ HLA-DR⁺ cells, the cells that are CD14⁻CD1c⁺CD1a⁺ and CD207⁺.

Thus, in a preferred embodiment, step a) of the method of the invention is performed by flow cytometry, preferably by measuring the expression of CD3, CD16, CD19, CD20, CD14, HLA-DR, CD1c, CD1a and CD207 biomarkers at the surface of the CD45⁺ leukocytes, in order to detect the frequency of the DC2+DC3 cells of the invention among CD45⁺ leukocytes.

In this particular embodiment, the use of fluorochrome agents attached to anti-CD45, anti-CD3, anti-CD16, anti-CD19, anti-CD20, anti-CD14, anti-CD1c, anti-CD207, anti-CD1a, anti-CADM1 and/or anti-CD141 antibodies to enable the flow cytometer to analyse on the basis of size, granularity and fluorescent light is highly advantageous. Thus, the flow cytometer can be configured to provide information about the relative size (forward scatter or “FSC”), granularity or internal complexity (side scatter or “SSC”), and relative fluorescent intensity of the cell sample. The fluorescent light determines CD45-expressing, CD3, CD16, CD19, CD20 non-expressing cells but HLA-DR, CD1c, CD207 and CD1a-expressing cells as being “CD207+CD1a+ dendritic cells” (DC2+DC3). In parallel, the fluorescent light determines CD45-expressing, CD3, CD16, CD19, CD20, CD1c, CD14 non-expressing but HLA-DR, CADM1, and CD141-expressing cells as being “dendritic cells type 1 (cDC1)”.

Preferably, the tumour core is determined by the surgeon during the surgery. In the context of the invention, it is possible to use conventional steps so as to prepare the tumour core that has been previously collected and stored under appropriate freezing conditions in order to be used by flow cytometry. In particular, the samples can be washed with appropriate buffers in order to separate the cells and permit the proper binding of the antibodies to the surface markers.

In the context of the present invention, the expression of the cell surface antigens may also be assessed using well known technologies such as cell membrane staining using biotinylation or other equivalent techniques followed by immunoprecipitation with specific antibodies, flow cytometry, western blot, ELISA or ELISPOT, antibodies microarrays, or tissue microarrays coupled to immunohistochemistry. Other suitable techniques include FRET or BRET, single cell microscopic or histochemistry methods using single or multiple excitation wavelength and applying any of the adapted optical methods, such as electrochemical methods (voltammetry and amperometry techniques), atomic force microscopy, and radio frequency methods, e.g. multipolar resonance spectroscopy, confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), cell ELISA, radioisotopic, magnetic resonance imaging, analysis by polyacrylamide gel electrophoresis (SDS-PAGE); HPLC-Mass Spectroscopy; Liquid Chromatography/Mass Spectrometry/Mass Spectrometry (LC-MS/MS)).

In another preferred embodiment, step a) of the method of the invention requires assessing the frequency or the density of CD207⁺ DC2+DC3 by immunohistochemistry on a sample of the tumour, preferably on a sample of the tumour core.

Immunohistochemistry is a well-known technique. In the context of the invention, it is possible to use conventional steps so as to fixate, dehydrate, embedded and section the tumour sample that has been previously collected and stored under appropriate freezing conditions. In particular, it is possible to preserve the tumour tissues embedded in paraffin (FFPE) and to perform histology directly on such samples.

Immunostainings can be performed on 3 μm thick whole sections prepared from FFPE blocks of tumour, as disclosed in the examples below.

The tumour core can be easily visually identified on the embedded tissue by an anatomo-pathologist following haematoxylin and eosin chemical staining.

In a preferred embodiment, the identification of the DC2+DC3 dendritic cells of the invention and the determination of their density is made by immunohistochemistry, automatically, by using an anti-CD207 antibody only. In another preferred embodiment, the method of the invention uses an anti-CD207 antibody and an anti-CLEC9A (and/or anti-XCR1) antibody, in order to automatically quantify by immunohistochemistry the density or the frequency of DC2+DC3 and of cDC1 cells, respectively, and be able to calculate the ratio of the invention (see below for more details).

In another preferred embodiment, step a) of the method of the invention is performed by measuring the expression level of the CD1A and CD207 genes in a nucleotide sample of the tumour, preferably in a RNA sample of the tumour core by measuring the quantity of CD1A and CD207 mRNAs present in same. It is also possible to measure the expression level of the HLA DQB2 gene in the nucleotide sample of the tumor (for example by quantifying the amount of HLA DQB2 mRNA), in order to confirm the result provided by the expression levels of the two other genes.

In the context of these methods, the term “nucleotide sample” means a sample containing a detectable amount of RNA extracted from the cells of interest. The nucleotide sample may be obtained from any tumour sample, and, in particular, from a biopsy of a tumour tissue. The method of the invention can include the steps consisting of obtaining a tumour sample (e.g., a tissue biopsy) from said subject and extracting the nucleotide fraction from said tumour sample.

The nucleotide fraction can be extracted using any known method in the state of the art. In particular, the skilled person well knows how to prepare a tumour sample (that has been previously collected and stored under appropriate freezing conditions) in order to be used in gene analysis. Usually, the samples are washed with appropriate buffers and put in a lysis buffer so as to isolate the RNA. RNA is preferably extracted from said sample by using a convenient commercial extraction protocol such as those proposed by MOBIO, Qiagen or Zymo.

As used herein, the term “tumour nucleotide sequence” designates the sequence of the oligonucleotides contained in a tumour sample. Preferably, this sample contains all the mRNAs present in the tumour sample.

A number of widely used procedures exist for detecting and determining the abundance of a particular mRNA in nucleotide samples: Northern blot analysis, nuclease protection assays (NPA), in situ hybridization, and reverse transcription-polymerase chain reaction (RT-PCR), next generation RNA sequencing (RNAseq), scRNAseq, etc. In the context of the invention, the expression level of the target genes is preferably measured by RT-qPCR or by RNAseq. RT-qPCR is a well-known technology whose conditions are thoroughly explained in the notice of commercial kits (SIGMA-ALDRICH, QIAGEN, . . . ). In the context of the invention, RNAseq is the preferred technology to assess the expression levels of the genes CD207 and CD1A in a nucleotide sample of the tumor.

As shown in the examples below, the level of CD207⁺ DC2+DC3 cells in the tumour sample can give a quantitative score of the CD8 T_{RM}-cells infiltration in the region of interest. Also, this level can be used to measure the clonal proliferation of CD8 T cells within the tumour, which is inversely correlated to the level of CD207 DC2+DC3 cells in the tumour sample.

In the prognosis methods of the invention, if the frequency of CD207⁺ DC2+DC3 is increased in a sample of the tumour core, preferably in the glandular structure of the tumour nest, as compared to the reference value, then this means that the prognosis of the subject is bad.

Conversely, if the frequency of CD207⁺ DC2+DC3 is decreased in a sample of the tumour core, preferably in the glandular structure of the tumour nest, as compared to the reference value, then this means that the prognosis of the subject is good.

By “bad prognosis”, it is herein meant that the outcome of the tested patient is likely to be a short survival, typically a survival of less than one year, two years, or five years. Patients having such Overall Survival is also called a “bad responder” if the patient was receiving a therapy. By “good prognosis”, it is herein meant that the outcome of the tested patient is likely to be a long survival, typically longer than 5 years, or more than 7 years.

According to the method of the invention, the absolute frequency of CD207⁺ DC2+DC3 present in the tumour sample of the subject may be used to determine the prognosis of said cancer. However, as shown on FIG. 3D, it can be more advantageous to normalize this value to the density or frequency of conventional dendritic cells of type 1 (cDC1) in the said sample, so as to get a reliable absolute value that has not to be compared to any reference value and can therefore be used without having access to any reference values. As shown by the results below, this ratio enables to precisely predict the T-cell clonality in the tumour.

In another aspect, the present invention encompasses a method to prognose the evolution of a solid cancer in a subject in need thereof, said method comprising the steps of:
a) Detecting and quantifying the quantity, density or frequency of CD207⁺ conventional dendritic cells of type 2 (DC2+DC3) cell population as defined above, in a tumour sample of said subject,
b) Detecting and quantifying the quantity, density or frequency of conventional dendritic cells of type 1 (cDC1) cells in the same tumour sample,
c) Comparing the value of step a) to the value obtained in step b), and
d) Prognosing that the subject has a bad prognosis if the quantity, density or frequency of CD207⁺ DC2+DC3 cell population is higher than the quantity, density or frequency of cDC1 cells.

The detection and quantification of the cells can be performed by any of the conventional means proposed above.

In a preferred embodiment, said steps a) and b) are performed by exclusion gating flow cytometry.

In this case, the CD207⁺ DC2+DC3 cells can be identified as proposed above (see also FIG. 6).

As for the cDC1 cells, they are dendritic cells expressing high level of CD141, CADM1, CLEC9A, XCR1, BTLA. All these markers are well known. By flow cytometry, cDC1 cells can be detected by identifying the CD45⁺CD3⁻ CD16⁻CD19⁻ CD20⁻ CD14⁻ CD1c⁻ CD141⁺ CADM1⁺ cells. In a particular embodiment, as explained in FIG. 6, the cDC1 cells can be identified by flow cytometry by selecting live cells within the tumour cells, then CD45⁺ cells, then, among these CD45⁺ cells, CD3⁻CD16⁻CD19⁻CD20⁻ cells that are also HLA-DR⁺, then, among these CD3⁻CD16⁻CD19⁻CD20⁻ HLA-DR⁺ cells, the cells that are CD14⁻CD1c⁻CD141⁺ and CADM1⁺. All these markers are well known.

The detection and quantification of the CD207⁺ DC2+DC3 cells and of the cDC1 cells can alternatively be performed by any of the conventional means described above.

In another preferred embodiment, said steps a) and b) are performed by immunohistochemistry. In this case, the CD207⁺ DC2+DC3 cells can be identified as proposed above. As for cDC1 cells, they can be easily identify by immunohistochemistry by using either anti-XCR1 antibodies, or anti-CLEC9A antibodies, or both antibodies.

In another preferred embodiment, said steps a) and b) are performed by RNAseq. As mentioned above, the quantification of the DC2+DC3 cells can also be done by measuring the mRNA levels of the CD207 gene and/or of the CD1A gene. In addition, the quantification of the cDC1 cells can be done by measuring the mRNA levels of the XCR1 gene and/or of the CLEC9A gene.

In an alternate embodiment, the steps c) and d) of this method are replaced by the calculation of a ratio between the quantity, density or frequency of CD207⁺ DC2+DC3 cells present in a sample of a solid tumour and the quantity, density or frequency of cDC1 cells present in said sample.

The ratio may involve the calculation of the density of CD207⁺ DC2+DC3 present in a sample of a solid tumour and the density of cDC1 cells present in said sample, both densities being detected by immunohistochemistry by using anti-CD207 antibodies (for detecting DC207⁺ DC2+DC3 cells) and anti-CLEC9A or antiXCR1 antibodies (for detecting cDC1 cells).

The ratio may involve the calculation of the frequency of CD207⁺ DC2+DC3 present in a sample of a solid tumour and the frequency of cDC1 cells present in said sample, both frequencies being detected by flow cytometry by using the antibodies mentioned above for detecting DC207⁺ DC2+DC3 cells and cDC1 cells by flow cytometry.

The ratio may involve the calculation of the amount of CD207⁺ DC2+DC3 present in a sample of a solid tumour and the amount of cDC1 cells present in said sample, both amounts being measured by RNAseq by measuring for example the mRNA level of the CD207 gene (for detecting DC207⁺ DC2+DC3 cells) and the mRNA level of the XCR1 gene (for detecting cDC1 cells).

If the ratio CD207⁺ DC2+DC3/cDC1 cells is superior or equal to 1, then the prognosis of said cancer is bad. If said ratio is inferior to 1, then the prognosis of said cancer is good.

Also, the value of this ratio allows to evaluate and predict the level of T-cell clonality in the tumour.

If the ratio CD207⁺ DC2+DC3/cDC1 cells is superior or equal to 1, then the T-cell clonality in said tumour is low. If said ratio is inferior to 1, then the T-cell clonality in said tumour is high.

In an alternate embodiment, the steps c) and d) are replaced by the calculation of a ratio between the quantity, density or frequency of cDC1 cells present in a sample of a solid tumour and the quantity, density or frequency of CD207⁺ DC2+DC3 cells present in said sample.

If the ratio cDC1/CD207⁺ DC2+DC3 is inferior to 1, then the prognosis of said cancer is bad. If said ratio is superior or equal to 1, then the prognosis of said cancer is good.

If the ratio cDC1/CD207⁺ DC2+DC3 is superior or equal to 1, then the T-cell clonality in said tumour is high. If said ratio is inferior to 1, then the T-cell clonality in said tumour is low.

Altogether, the present invention targets the use of the ratio between the quantity, density or frequency of CD207⁺ DC2+DC3 cells present in a sample of a solid tumour and the quantity, density or frequency of cDC1 cells present in the same sample of solid tumour (or vice versa) to prognose the evolution of a solid cancer or the T-cell clonality in the tumour.

This frequency is preferably obtained by exclusion gating flow cytometry, whereas the density is preferably obtained by immunohistochemistry and the quantity of the cells by RNAseq.

By “bad prognosis”, it is herein meant that the outcome of the tested patient is likely to be a short survival, typically a survival of less than one year, two years, or five years. Patients having such Overall Survival is also called a “bad responder” if the patient was receiving a therapy. By “good prognosis”, it is herein meant that the outcome of the tested patient is likely to be a long survival, typically longer than 5 years, or more than 7 years.

### Methods for Selecting Treatments for Solid Tumours

The method of the invention can be used to predict the outcome of cancer patients. In a preferred embodiment, it is used to aid the skilled cancerologist in the selection of appropriate treatments for maximizing the survival of the patients. Appropriate treatments are for example chemotherapeutic treatments, immunotherapeutic treatments, radiotherapeutic treatments and/or surgery. Preferably, the signature of the invention is generated before initiating a treatment.

Specifically, said patients have been treated or will be treated with anti-cancer drugs as defined below.

The present prognostic tool may also assist physicians in identifying patients who are likely to progress towards even more serious form of solid cancer and thus may suggest those patients require heavier or more aggressive treatment.

The methods of the invention furthermore enable to generate a personalized treatment plan. The personalized treatment plan is based on the high or low level of the cells of the invention in the tumour sample. The personalized treatment plan may include a new therapeutic recommendation, a new therapeutic schedule, a new therapeutic dosage, a follow up treatment schedule, or other action. The personalized treatment plan may include information that facilitates developing a precision treatment plan for a patient. For example, upon determining that a tumour is classified as a responder, the method of the invention may control the personalized cancer treatment system to generate a first personalized treatment plan that indicates a first type of therapy. Upon determining that the tumour is classified as a non-responder to this first therapy, the method may generate a second, different personalized treatment plan that proposes a second, different type of therapy.

By increasing the accuracy with which response to a therapy is predicted, the methods of the invention produce the concrete, real-world technical effect of reducing the amount of unnecessary biopsies or other invasive procedures for patients who are unlikely to benefit from immunotherapy treatment. Additionally, these methods reduce the expenditure of time, money and therapeutic resources on patients who are unlikely to benefit from the treatment. They thus improve on conventional approaches to predicting response to therapy in a measurable, clinically significant way.

In a preferred embodiment, the methods of the invention can be used:
to evaluate the response of a patient to an anti-cancer treatment,
to select an anti-cancer therapy for a patient,
to assess the efficacy of an anti-cancer therapy for a patient,
to adapt an anti-cancer therapy for a patient and/or
to identify if a tumour or a patient is responding to the therapy,
generate a personalized treatment plan.

In one embodiment, the invention pertains to in vitro methods for selecting an anti-cancer therapy for a patient suffering from a solid cancer, said methods comprising the steps of:
a) performing the prognostic methods of the invention, as defined above, before and after a candidate anti-cancer therapy has been administered to the patient,
b) selecting said therapy if the level of CD207⁺ DC2+DC3 cells has significantly decreased or if the ratio between CD207⁺ DC2+DC3 cells and cDC1 cells has diminished, after the therapy was given.

In one embodiment, the invention pertains to in vitro methods for assessing the efficacy of an anti-cancer therapy in a patient suffering from a solid cancer, said methods comprising the steps of:
a) performing the prognostic methods of the invention, as defined above, before and after a candidate anti-cancer therapy has been administered to the patient, and
b) concluding that said therapy is efficient or that the tumour is responding to said therapy if the level of CD207⁺DC2+DC3 cells has significantly decreased or if the ratio between CD207⁺ DC2+DC3 cells and cDC1 cells has diminished, after the therapy was given, or
c) concluding that said therapy is not efficient or that the tumour is not responding to said therapy if the level of CD207⁺ DC2+DC3 cells has significantly increased or if the ratio between CD207⁺ DC2+DC3 cells and cDC1 cells has increased, after the therapy was given.

In one embodiment, the invention pertains to in vitro methods for adapting an anti-cancer therapy in a patient suffering from a solid cancer, said methods comprising the steps of:
a) performing the prognostic methods of the invention, as defined above, before and after a candidate anti-cancer therapy has been administered to the patient,
b) concluding that said therapy is efficient or that the tumour is responding to said therapy if the level of CD207⁺DC2+DC3 cells has significantly decreased or if the ratio between CD207⁺ DC2+DC3 cells and cDC1 cells has diminished, after the therapy was given, and maintaining same,
c) concluding that said therapy is not efficient or that the tumour is not responding to said therapy if the level of CD207⁺ DC2+DC3 cells has significantly increased or if the ratio between CD207⁺ DC2+DC3 cells and cDC1 cells has increased, after the therapy was given, and therefore changing same.

Thus, said adaptation of the anti-cancer therapy may consist in:
the continuation, a reduction or suppression of the said therapy if the therapy has been assessed as efficient, or adapted to said “responder” tumour, or if the prognosis is sufficiently good.
an augmentation of the said therapy or a change to a more aggressive therapy, if said therapy of step a) has been assessed as non-efficient or non-adapted to said “non-responder” tumour.

In the context of the invention, the response to a therapy is preferably defined according to RECIST 1.1 criteria. A Complete Response (CR) is defined as a disappearance of all target lesions. Any pathological lymph nodes (whether target or non-target) must have reduction in short axis to <10 mm. A Partial Response (PR) is defined as at least a 30% decrease in the sum of diameters of target lesions, taking as reference the baseline sum diameters. A Progressive Disease (PD) is defined as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). A Stable Disease (SD) is defined as neither sufficient shrinkage to qualify for PR nor sufficient increase to qualify for PD, taking as reference the smallest sum diameters while on study.

Time of the evaluation of the resistance or response to the therapy often depends of the disease (it is usually comprised between 6 weeks and 3-6 months). A “non-responder” is considered as a patient with a progression disease or a stable disease as defined according to RECIST 1.1 criteria.

Also, the present invention targets the use of the ratio between the amount, the density or the frequency of CD207⁺ DC2+DC3 cells present in a sample of a solid tumour and the amount, the density or the frequency of cDC1 cells present in the same sample of solid tumour to evaluate the response of a patient to an anti-cancer treatment, to select an anti-cancer therapy for a patient, to assess the efficacy of an anti-cancer therapy for a patient, to adapt an anti-cancer therapy for a patient, to identify if a tumour or a patient is responding to the therapy, or to generate a personalized treatment plan, as explained above.

### Method for Treating Solid Tumours by CDC, ADC or ADCC

In a final aspect, the present inventors propose to target cytotoxic agents to the CD207⁺ DC2+DC3 cells in order to deplete or destroy them and to increase the level of T CD8⁺ within the tumour, thereby increasing the prognosis of the patients and eventually treat them.

Said agent can be, for example, bicyclic peptides, antibodies, or antibody fragments like diabodies, Fab, or scFV.

In a particular embodiment, the present invention proposes a cytotoxic antibody that specifically binds to CD207⁺ DC2+DC3 cells of the invention, for use for depleting said cells from the tumour core of a solid tumour.

This antibody is herein called “antibody of the invention”. It can be a chimerized or a humanized antibody, as defined below. It can be multispecific, and in particular bispecific. As such, it can be chosen in the group consisting of: bispecific IgGs, IgG-scFv2, (scFv)4-IgG, (Fab′)2, (scFv)2, (dsFv)2, Fab-scFv fusion proteins, (Fab-scFv)2, (scFv)2-Fab, (scFv-CH2-CH3-scFv)2, bibody, tribody, bispecific diabody, disulfide-stabilized (ds) diabody, ‘knob-into whole’ diabody, single-chain diabody (scDb), tandem diabody (TandAb), flexibody, DiBi miniantibody, [(scFv)2-Fc]2, (scDb-CH3)2, (scDb-Fc)2, Di-diabody, Tandemab., etc.

Preferably, the cytotoxic agent of the invention is an antibody that specifically binds to the CD207 protein located at the surface of CD207⁺ DC2+DC3. Bispecific antibodies targeting the CD207 protein can also be used. Bispecific antibodies targeting the CD1a and the CD207 (Langerin) protein can also be used. This cytotoxic antibody would preferably bind with high affinity and specificity dendritic cells that express high levels of CD1a and CD207 biomarkers. These biomarkers have been described above.

Preferably, the antibodies of the invention have a high affinity for CD207. More preferably, they possess a very low dissociation constant with these receptors. For example, a low dissociation constant is inferior or equal to 50 nM and may reach down to the picomolar range (10⁻¹² M). More specifically, the antibodies of the invention or antigen-binding fragments thereof have a dissociation constant (K_{D}) with CD207 comprised between about 5 nM and about 20 nM as measured by Surface Plasmon Resonance.

The antibodies of the invention would be preferably conjugated to a potent cytotoxic compound such as a radioisotope, a chemotherapeutic drug or a toxin, so as to provide an “Antibody-Drug Conjugate” (ADC). Examples thereof are given below.

In particular embodiments, the antibodies of the invention comprise changes in amino acid residues in the Fc region that lead to improved effector function including enhanced complement-dependent cytotoxicity (CDC) and/or antibody-dependent cellular cytotoxicity (ADCC) function and eventually DC-cell killing (also referred to herein as DC-cell depletion). In particular, three mutations have been identified for improving CDC and ADCC activity: S298A/E333A/K334A (also referred to herein as a triple Ala mutant or variant; numbering in the Fc region is according to the EU numbering system). This may be achieved by introducing one or more amino acid substitutions in an Fc region of an antibody.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, monocytes and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express Fc[gamma]RIII only, whereas monocytes express Fc[gamma]RI, Fc[gamma]RII and Fc[gamma]RIII. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. (USA) 95:652-656 (1998). To promote ADCC, cysteine residue(s) may be introduced in the Fc region of the antibodies of the invention, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), can be performed.

Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164:4178-4184 (2000).

In particular embodiments, the antibody of the invention would be conjugated to a potent cytotoxic drug so as to mediate ADC, or would be modified so as to mediate efficient ADCC or CDC, as detailed above.

This cytotoxic antibody would be administered intratumourally in a sufficient amount for treating solid cancer in a subject in need thereof or for preventing metastasis to develop.

### DEFINITIONS

Unless specifically defined, all technical and scientific terms used herein have the same meaning as commonly understood by a skill artisan in chemistry, biochemistry, cellular biology, molecular biology, and medical sciences.

The “frequency” of a particular cell population in a given sample is herein understood as being the proportion of this particular cell population among the cells present in said sample. It can be measured by calculating the percentage of cells of this particular population (i.e., displaying the markers known to be shared by the cells of this population) present in said sample, among the total number of cells present in the tested sample, or among the cells of another particular cell population (e.g., in the context of the invention, among all DC cells or among CD45⁺ leukocytes). It can also be the number of cells belonging to the target population divided by the number of other cells, provided that said number of other cells is normalized between samples, so as to be comparable. In that sense, the “frequency” of the cells of the invention can be herein assimilated to the “concentration” or the “abundance” of the cells belonging to the target population of the invention, within a particular category of cells. The term “frequency” as meant herein is therefore synonymous of the terms “proportion”, “percentage” or “concentration” which can be used interchangeably.

A “subject” which may be subjected to the methods described herein may be any of mammalian animals including human, dog, cat, cattle, goat, pig, swine, sheep and monkey. More preferably, the subject of the invention is human subject; a human subject can be known as a patient. In one embodiment, “subject” or “subject in need” refers to a mammal, preferably a human, that suffers from a solid cancer or is suspected of suffering from a solid cancer or has been diagnosed with solid cancer. A “control subject” refers to a mammal, preferably a human, which is not suffering from a solid cancer, and is not suspected of suffering from solid cancer.

“Prognosis” herein means the prediction/determination/assessment of the risk of disease (in particular a cancer and/or a tumour) progression (or evolution, or development) in an individual. Prognosis includes the assessment of the future development of the subject's condition and the possible chances of cure. The prognosis can be determined on the basis of observations and/or measurements, carried out using various tools.

As used herein, “stratification” refers to the separation/classification of subjects into subgroups by disease (in particular a cancer and/or a tumour) severity. The different subgroups include the subgroup of healthy subjects as well as different subgroups of subjects with a disease, classified according to the stage of disease progression/advancement/severity (e.g., WHO grade I, II, III or IV cancer; and/or metastatic or non-metastatic brain cancer).

“Monitoring” or “Follow-up” herein refers to the identification/assessment of the progression (or evolution, or development) of a disease (in particular a cancer and/or a tumour) in a subject. Monitoring may be carried out on the basis of observations and/or measurements, using different tools, at different time intervals. Intervals may be regular or irregular. Their frequency depends on the cancer but also on the stage of cancer progression. It can range from a few days (e.g. in case of severe/advanced/severe disease and/or rapidly progressing cancer and/or exacerbation phase) to a few years (e.g. in case of early, mild or moderate cancer and/or slowly progressing cancer).

As used herein, “determining the outcome” or “outcome determination”, herein means the prediction/determination/assessment of the most probable evolution (or progression or development) of a cancer (in particular a cancer and/or a tumour) in a subject. “Determining the outcome” of the disease thus includes at least assessing the next stages that are most likely to be undergone by the subject, in terms of probability. More specifically, “determining the outcome of a solid tumour” includes, but is not limited to, the assessment of the probabilities (or chances), for a subject of switching to a metastatic form; and/or the assessment of the probabilities (or chances), for a subject of having or progressing towards a tumour and/or a metastatic tumour.

As used herein, “predicting the metastasis-associated risk” or “assessing the risk of switching to a metastatic form” means the prediction/determination/assessment of the probabilities (or the chances) of exacerbation (or aggravation, or intensification) of a cancer and/or a tumour, in particular the prediction/determination/assessment of the probabilities (or the chances) to switch from a non-metastatic to a metastatic form.

As used herein, the terms “treat”, “treating”, “treatment”, and the like refer to reducing or ameliorating the symptoms of a disorder (e.g., solid cancer). It will be appreciated that, although not precluded, treating a disorder or condition does not require that the disorder, condition or symptoms associated therewith be completely eliminated. As used herein “treating” a disease in a subject or “treating” a subject having a disease refers to subjecting the subject to a pharmaceutical treatment, e.g., the administration of a drug, such that the extent of the disease is decreased or prevented. For examples, treating results in the reduction of at least one sign or symptom of the disease or condition. Treatment includes (but is not limited to) administration of a composition, such as a pharmaceutical composition, and may be performed either prophylactically, or subsequent or the initiation of a pathologic event. Treatment can require administration of an agent and/or treatment more than once.

As used herein, “selecting a therapy” or “selecting a treatment” or “selecting a drug” refers to the process of selecting (choosing, or deciding for, or opting for) the most appropriate therapy for a subject, in view of the symptoms (or signs) detected (observed and/or measured) in the subject, and/or in view of the subject himself, and the general knowledge in the medical field (preferably the medical field closest to the disease). Selecting a therapy include selecting the most appropriate therapy and may include also selecting the most appropriate administration mode and/or the most appropriate posology.

As used herein, the terms “assessing the efficacy of a therapy” or “assessment of treatment efficacy” refers to the determination of the clinical status of a subject undergoing treatment (i.e. receiving a therapy). The treatment may be preventive, for example in the case of predisposition to a disease, or it may be curative, for example in the case of a diagnosed disease. For example, the effectiveness of the treatment can be assessed by determining the condition of the subject at different time intervals. In particular, the condition of the subject can be assessed before the first dose of treatment and then at regular (or irregular) intervals after the first dose (e.g. after each new dose of treatment). A comparison of the subject's condition at these different intervals can then be made to identify any changes. The patient's condition can be assessed on the basis of observations and/or measurements made with different tools.

The “solid tumour” or “solid cancer” to be prognosed or treated by means of the methods of the invention can be metastatic or not, notably selected in the group consisting of: squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, brain cancer, central nervous system cancer, pancreatic cancer, glioblastoma multiforme, cervical cancer, stomach cancer, bladder cancer, malignant hepatoma, breast cancer, colon carcinoma, head and neck cancer, gastric cancer, germ cell tumor, pediatric sarcoma, rhabdomyosarcoma, Ewing's sarcoma, osteosarcoma, soft tissue sarcoma, sinonasal NK/T-cell lymphoma, myeloma, melanoma, multiple myeloma. In particular, these solid tumours can be specifically lung cancer, malignant mesothelioma, bladder cancer, kidney cancer, testicular cancer, breast cancer, cancer of the upper aero-digestive tract, liver cancer, pancreas cancer, stomach cancer; colon cancer or ovarian cancer. More preferably, said solid tumour is a lung adenocarcinoma, such as a Non-Small Cell Lung Cancer. More preferably, said solid tumour is a breast cancer, such as triple-negative breast cancer (TNBC).

In a particular embodiment, the solid tumour or solid cancer to be prognosed or treated by means of the methods of the invention is chosen in the group consisting of: Bladder Carcinoma, Breast cancer, Cervical squamous cell carcinoma, Esophageal Adenocarcinoma, Esophageal Squamous Cell Carcinoma, Gastric cancer, Glioblastoma, Head-neck squamous cell carcinoma, Kidney renal clear cell carcinoma, Kidney renal papillary cell carcinoma, Liver hepatocellular carcinoma, Lung adenocarcinoma, Non-Small Cell Lung Cancer, Lung squamous cell carcinoma, Ovarian cancer, Pancreatic ductal adenocarcinoma, Pheochromocytoma and Paraganglioma, Rectum adenocarcinoma, Renal Cancer, Sarcoma, Stomach adenocarcinoma, Testicular Germ Cell Tumor, Thymoma, Thyroid carcinoma, Uterine corpus endometrial carcinoma and metastatic Urothelial cancer.

In a particular embodiment, the solid tumour or solid cancer to be prognosed or treated by means of the methods of the invention is not a melanoma.

As used herein, the term “anti-cancer treatment” or “anti-cancer therapy” or “therapy” designates any chemical or biochemical drug that can be used to treat a solid cancer. It is meant a substance which, when administered to a patient, treats or prevents the development of cancer in the patient. By way of non-limiting example for such agents, mention may be made of an antitumor/cytotoxic antibiotic, alkylating agents, antimetabolites, a topoisomerase inhibitor, a mitotic inhibitor, a platin based component, a specific kinase inhibitor, a hormone, a cytokine, an antiangiogenic agent, an antibody, a DNA methyltransferase inhibitor, a cancer vaccine, an immune checkpoint blocker and a vascular disrupting agent.

Said antitumor agent or cytotoxic antibiotic can for example be selected from an anthracycline (e.g. doxorubicin, daunorubicin, adriamycine, idarubicin, epirubicin, mitoxantrone, valrubicin), actinomycin, bleomycin, mitomycin C, plicamycin and hydroxyurea.

Said alkylating agent can for example be selected from mechlorethamine, cyclophosphamide, melphalan, chlorambucil, ifosfamide, temozolomide busulfan, N-Nitroso-N-methylurea (MNU), carmustine (BCNU), lomustine (CCNU), semustine (MeCCNU), fotemustine, streptozotocin, dacarbazine, mitozolomide, thiotepa, mytomycin, diaziquone (AZQ), procarbazine, hexamethylmelamine and uramustine.

Said antimetabolite can for example be selected from a pyrimidine analogue (e.g. a fluoropyrimidine analog, 5-fluorouracil (5-FU), floxuridine (FUDR), cytosine arabinoside (Cytarabine), Gemcitabine (Gemzar®), capecitabine); a purine analogue (e.g. azathioprine, mercaptopurine, thioguanine, fludarabine, pentostatin, cladribine, clofarabine); a folic acid analogue (e.g. methotrexate, folic acid, pemetrexed, aminopterin, raltitrexed, trimethoprim, pyrimethamine).

Said topoisomerase inhibitor can for example be selected from camptothecin, irinotecan, topotecan, amsacrine, etoposide, etoposide phosphate and teniposide.

Said mitotic inhibitor can for example be selected from a taxane [paclitaxel (PG-paclitaxel and DHA-paclitaxel) (Taxol®), docetaxel (Taxotère®), larotaxel, cabazitaxel, ortataxel, tesetaxel, or taxoprexin]; a spindle poison or a vinca alkaloid (e.g. vincristine, vinblastine, vinorelbine, vindesine or vinflunine); mebendazole; and colchicine.

Said platin based component can for example be selected from platinum, cisplatin, carboplatin, nedaplatin, oxaliplatin, satraplatin and triplatin tetranitrate.

Said specific kinase inhibitor can for example be selected from a BRAF kinase inhibitor such as vemurafenib; a MAPK inhibitor (such as dabrafenib); a MEK inhibitor (such as trametinib); and a tyrosine kinase inhibitor such as imatinib, gefitinib, erlotinib, sunitinib or carbozantinib.

Tamoxifen, an anti-aromatase, or an anti-estrogen drug can also typically be used in the context of hormonotherapy.

A cytokine usable in the context of an immunotherapy can be selected for example from IL-2 (Interleukine-2), IL-11 (Interleukine-11), IFN (Interferon) alpha (IFNa), and Granulocyte-macrophage colony-stimulating factor (GM-CSF).

Said anti-angiogenic agent can be selected for example from bevacizumab, sorafenib, sunitinib, pazopanib and everolimus.

Said antibody, in particular the monoclonal antibody (mAb) can be selected from a anti-CD20 antibody (anti-pan B-Cell antigen), anti-Her2/Neu (Human Epidermal Growth Factor Receptor-2/NEU) antibody; an antibody targeting cancer cell surface (such as rituximab and alemtuzumab); a antibody targeting growth factor (such as bevacizumab, cetuximab, panitumumab and trastuzumab); a agonistic antibody (such as anti-ICOS mAb, anti-OX40 mAb, anti-41BB mAb); and an immunoconjugate (such as 90Y-ibritumomab tiuxetan, 131I-tositumomab, or ado-trastuzumab emtansine).

Said DNA methyltransferase inhibitor can for example be selected from 2′-deoxy-5-azacytidine (DAC), 5-azacytidine, 5-aza-2′-deoxycytidine, 1-[beta]-D-arabinofuranosyl-5-azacytosine and dihydro-5-azacytidine.

Said vascular disrupting agent can for example be selected from a flavone acetic acid derivative, 5,6-dimethylxanthenone-4-acetic acid (DMXAA) and flavone acetic acid (FAA).

Other chemotherapeutic drugs include a proteasome inhibitor (such as bortezomib), a DNA strand break compound (such as tirapazamine), an inhibitor of both thioredoxin reductase and ribonucleotide reductase (such as xcytrin), an immune checkpoint blocker and an enhancer of the Thl immune response (such as thymalfasin).

Said immune checkpoint blocker is typically an antibody targeting an immune checkpoint. Such an immune checkpoint blocker can be advantageously selected from anti-CTLA4 (ipilimumab and Tremelimumab), anti-PD-1 (Nivolumab and Pembrolizumab), anti-PD-L1 (Atezolizumab, Durvalumab, and Avelumab), anti-PD-L2 and anti-Tim3 or combinations thereof. Specifically, said patients have been treated or will be treated with immunotherapy drugs such as anti-PD-1 and/or anti-PD-L1 drugs.

Said cancer vaccine can for example be selected from a vaccine composition comprising (antigenic) peptides; a Human papillomavirus (HPV) vaccine (such as Gardasil®, Gardasil9®, and Cervarix®); a vaccine stimulating an immune response to prostatic acid phosphatase (PAP) sipuleucel-T (Provenge®); an oncolytic virus and talimogene laherparepvec (T-VEC or Imlygic®).

The treatment which can include several anticancer agents is selected by the cancerologist depending on the specific cancer to be prevented or treated.

The term “anti-cancer treatment” or “anti-cancer therapy” or “therapy” also designates any other treatment that was proven beneficial for treating cancer, namely radiotherapy, immunotherapy, or surgery. The radiotherapy typically involves rays selected from X-rays (“XR”), gamma rays and/or UVC rays.

“Dendritic cells” (DCs) are professional antigen-presenting cells that link innate and adaptive immunity and are critical for the induction of protective immune responses against pathogens. Their main function is to process antigen material and present it on the cell surface to the T cells of the immune system. They act as messengers between the innate and the adaptive immune systems. Dendritic cells are present in those tissues that are in contact with the external environment, such as the skin (where there is a specialized dendritic cell type called the Langerhans cell) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites that give the cell its name.

“Granulocytes” are a type of leukocytes characterized by the presence of granules in their cytoplasm. The types of these cells are neutrophils, eosinophils, and basophils. “T cells” or “T lymphocytes” are a type of lymphocyte that plays a central role in cell-mediated immunity. They can be distinguished from other lymphocytes, such as B cells and natural killer cells (NK cells), by the presence of a T-cell receptor (TCR) on the cell surface. “B cells” or “B lymphocytes” are a type of lymphocyte in the humoral immunity of the adaptive immune system. They can be distinguished from other lymphocytes, such as T cells and natural killer cells (NK cells), by the presence of a protein on the B cells outer cell surface known as a B-cell receptor (BCR). “Natural killer cells” (or “NK cells”) are a type of cytotoxic lymphocytes that kill cells by releasing small cytoplasmic granules of proteins called perforin and granzyme. They constitute the third kind of cells differentiated from the common lymphoid progenitor generating B and T lymphocytes.

According to the present invention, a cell “expresses CD207” (or CD1a or any other biomarker) if CD207 (or CD1a or any other biomarker) is present at a significant level on its surface (such a cell being also defined as a “CD207⁺ cell” (or a “CD1a⁺ cell”, etc.). In particular, a cell expresses CD207 (or CD1a or any other biomarker) if the signal associated to surface CD207 (or CD1a or any other biomarker) staining (e.g. obtained with an antibody anti-CD207 coupled to a fluorescent marker) which is measured for said cell is superior to the signal corresponding to the staining of one cell being known as not expressing CD207 (or CD1a or any other biomarker). In a preferred embodiment, CD207⁺ cells (or CD1a⁺ cells, etc.) are such that the ratio between the surface CD207− (or CD1a− or any other biomarker)-associated signal measured for said cells and the surface CD207− (or CD1a− or said other biomarker)-associated signal measured for cells being known as expressing CD207 (or CD1a or said other biomarker) is superior or equal to 1. Cells expressing CD207 (or CD1a or said other biomarker) at their surface are well known in the art.

The term “reference value”, as used herein, refers to the expression level of a prognosis marker under consideration in a reference sample. A “reference sample”, as used herein, means a solid cancer sample obtained from subjects, preferably two or more subjects, known to be suffering from solid cancer with a good prognosis. The suitable reference expression levels can be determined by measuring the expression levels of said prognosis marker in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. It can also be based on a sample from the subject being tested, taken from a non-cancerous tissue (i.e., a normal tissue of the same subject, adjacent to the tumour or not). The reference value can be based on a large number of samples, such as from population of subjects of the chronological age matched group, or based on a pool of samples including or excluding the sample to be tested.

The term “antibody” as used herein is intended to include monoclonal antibodies, polyclonal antibodies, and chimeric antibodies. Antibody fragments can also be used in the present diagnosis method. This term is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques. The antibodies used in the method of the invention can be of different isotypes (namely IgA, IgD, IgE, IgG or IgM). They may be from recombinant sources and/or produced in transgenic animals. Conventional techniques of molecular biology, microbiology and recombinant DNA techniques are within the skill of the art. Such techniques are explained fully in the literature. Commercial antibodies recognizing specifically the antigens expressed by tumour cells can be furthermore used. Some of them are listed in the experimental part below (said list being however not exhaustive nor limitating).

When used for prognosis purposes, antibodies can be detected by direct labeling with detectable markers. Alternatively, unlabeled primary antibody can be used in conjunction with a labeled secondary antibody, comprising antisera, polyclonal antisera or a monoclonal antibody specific for the primary antibody. Immunohistochemistry protocols and kits are well known in the art and are commercially available. In a preferred embodiment of the invention, these antibodies are tagged with a detectable marker, preferably a fluorescent or a luminescent marker. Examples of detectable markers/labels include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase examples of suitable prosthetic group complexes include streptavidin/biotin and avidin/biotin, examples of suitable fluorescent materials include umbelliferone, fluorescein, fluorescein isothiocyanate, rhodamine, dichlorot[pi]azinylamine fluorescein, dansyl chloride or phycoerythrin, an example of a luminescent material includes luminol, examples of bioluminescent materials include luciferase, luciferin, and aequorin, and examples of suitable radioactive material include ¹²⁵I, ¹³¹I, ³⁵S or ³H.

It must be understood here that the invention preferably does not relate to antibodies in natural form, i.e., they are not taken from their natural environment but are isolated or obtained by purification from natural sources or obtained by genetic recombination or chemical synthesis and thus they can carry “unnatural” amino acids as will be described below. They can also be multispecific, for example TandAb or Flexibody.

When used for therapeutic purposes, the antibodies can be chimeric or humanized, or antigen-binding fragments which can be obtained by genetic engineering or by chemical synthesis.

The term “chimeric antibody” as used herein refers to an antibody containing a natural variable region (light chain and heavy chain) derived from an antibody of a given species in combination with constant regions of the light chain and of the heavy chain of an antibody of a species heterologous to said given species. Thus, a “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Such chimeric antibodies, or fragments of same, can be prepared by recombinant engineering. For example, the chimeric antibody could be produced by cloning recombinant DNA containing a promoter and a sequence coding for the variable region of a non-human monoclonal antibody of the invention, notably murine, and a sequence coding for the human antibody constant region. A chimeric antibody according to the invention coded by one such recombinant gene could be, for example, a mouse-human chimera, the specificity of this antibody being determined by the variable region derived from the murine DNA and its isotype determined by the constant region derived from human DNA.

As used herein, the term “humanized antibody” refers to a chimeric antibody which contain minimal sequence derived from non-human immunoglobulin. In one embodiment, a humanized antibody is a human immunoglobulin (recipient antibody) in which residues from a CDR of the recipient are replaced by residues from a CDR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the Heavy (H) chain, and in the Light (L) chain, no more than 3. The humanized antibody optionally will also comprise at least a portion of an immunoglobulin constant region (Fc), typically that of a human immunoglobulin.

### FIGURE LEGENDS

FIG. 1. Spectral flow cytometric analysis of antigen presenting DC populations in 8 NSCLC patients.

(A) UMAP of singlet, live, CD45⁺ cells from normal adjacent and tumour samples of 4 representative NSCLC patients. (B) UMAP of extracted MNPs showing delineation of cDC1s, DC2+DC3s, CD123⁺ DCs (pDC and pre-DC) and monocytes/macrophages (MoMac). (C-D) Identification and quantification of cDC2s and DC3s and (E) DC3/cDC2 ratio between normal adjacent and tumour samples. (F) RNA expression of CD207 and CD1A and protein expression of CD103 on the DC-VERSE. (G-H) Identification and percentage of CD103⁺ “LTB” DC2+DC3s among total DC2+DC3s. (I-J) Identification and percentage of CD207⁺ DC2+DC3s among total DC2+DC3s. P-values were calculated using a paired t-test.

FIG. 2. Spatial analysis of CD207 DC2+DC3s

(A) the spatial mapping of CD207⁺ cells (CD207⁺ DC2+DC3s) of the same tumour cross-section are shown. (B) Density of CD207⁺ DC2+DC3s in normal adjacent (Healthy) lung and in the tumour (Tumour) regions from the 16 NSCLC tumours analysed. (C). H&E stainings (upper panels), the multiplexed fluorescent images (middle panels) are shown. The yellow dotted line delineates the tumour glandular areas from the tumour stroma. (D) Density of CD207⁺ DC2+DC3s and of CD3⁺CD8⁺ T-cells from 13 of the 16 NSCLC tumours that could be analysed for tumour vs stroma regions. P-values were calculated using the Wilcoxon paired non-parametric test in (B) and (D).

FIG. 3. Characterisation of the pathophysiological involvement of CD207 DC2+DC3s in human cancer patients.

(A) Percentage of predicted mega-clusters by Azimuth of query data from breast cancer patients (Bassez et al., 2021) categorised by T-cell clonality and treatment status (anti-PD-1 therapeutic monoclonal antibody=Immune Checkpoint Blocker=ICB). (B) cDC1 to CD207 DC2+DC3 ratio for patients from Bassez et al., with at least one CD207 DC2+DC3. (C) Pearson correlations between the percentage of CD207 DC2+DC3s and total CD8⁺ T-cells (left), CD8⁺ T_{RMs} (middle) or CD8⁺ T_{EMRAs} (right). (D) Pearson correlation between CD207 DC2+DC3s and CD8⁺ T-cells from flow cytometry analysis of tumours cells obtained from 8 NSCLC patients. (E) Correlation map of DC population signatures (defined in the DC-VERSE) and of other signatures obtained from Ramos et al., (Nalio Ramos et al., 2022). (F) Percentage of the CD207 DC2+DC3 signature-positive patients between Disease-Specific Survival (DSS) status in the seven TCGA cancer datasets. Deceased represents disease-specific death, and alive denotes disease-specific survival. [Breast Invasive Carcinoma (BRCA), Kidney Renal Clear Cell Carcinoma (KIRC), Lung Adenocarcinoma (LUAD), Lung Squamous Cell Carcinoma (LUSC), Thyroid Carcinoma (THCA), Lymphoid Neoplasm Diffuse Large B-cell Lymphoma (DLBC), Uterine Corpus Endometrial Carcinoma (UCEC)]. P-values were calculated using a Wilcoxon non-parametric paired test. Correlations were evaluated using the Pearson correlation (r) with two-tailed p-values. (G) Kaplan-Meier plot of the overall survival (OS) of patients with different cancers whose tumour was sampled and analysed by bulk RNAseq prior to immune checkpoint blockade (ICB) treatment. Patients were separated based on high or low expression of DC populations mean gene signatures (left and middle panels) or based on the ratio of XCR1 to CD207 transcripts (right panel). (H) Expression of CD207+CD1A (Fragments Per Kilobase per Million) in patients with different cancer types, depending on their response to the following ICB treatments: anti-PD1 monoclonal antibody for metastatic urothelial cancer, and, for the other cancer types: #1: cf. SRP183455, SRP217040; #2: anti-PD1; #3: cf. SRP217040; #4: cf. SRP183455; #5: cf. ERP105482, SRP070710, SRP094781, SRP150548, SRP230414, SRP250849, SRP302761; #6: cf. IMvigor210; #7: cf. ERP107734; #8: cf. SRP155030; #9: cf. SRP128156; (white bar: patient responding; black bar: patient non-responding).

FIG. 4.

(A) Percentage of CD207 DC2+DC3s between patients with non-expanded and expanded T-cell clonality. (B) Correlation between the frequencies of CD207 DC2+DC3s and CD8 T_{RMs} among CD45⁺ cells in lung tumours within the Leader et al. scRNAseq data (Leader et al., 2021). (C) Correlation between the frequencies of CD207⁺ DC2+DC3s and CD4⁺ T-cells from flow cytometry analysis of 8 NSCLC patients. The percentage of CD207 DC2+DC3s was graphed in both CD8⁺ T_{RM}^{hi} and CD8⁺ f^{lo} populations. (D) Histogram depicting the mean CD207 DC2+DC3 signature used for the LUAD TCGA cohort. The gate defining CD207 DC2+DC3 signature-positive patients is shown. Correlations were evaluated using the Pearson correlation (r) with two-tailed p values. P values were calculated using a t-test.

FIG. 5.

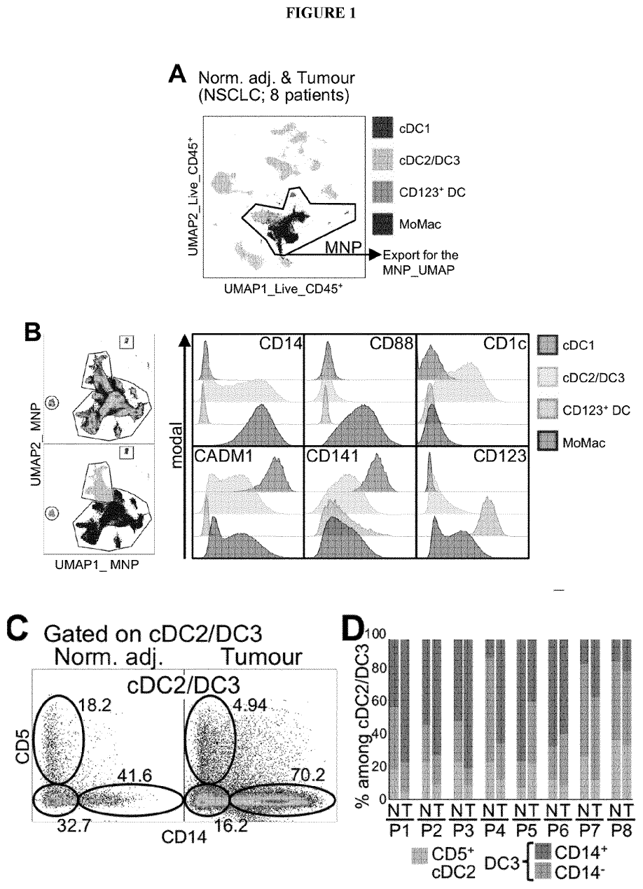

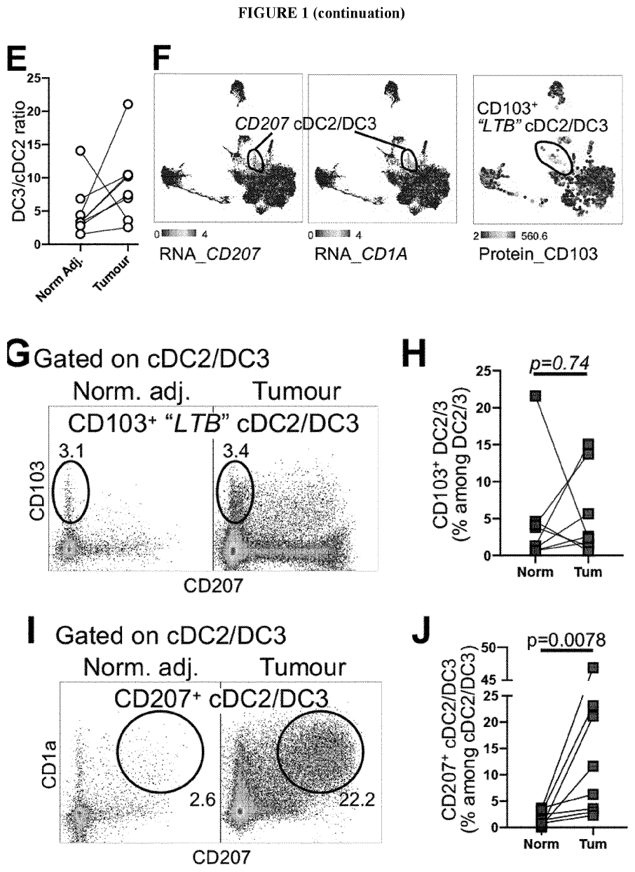

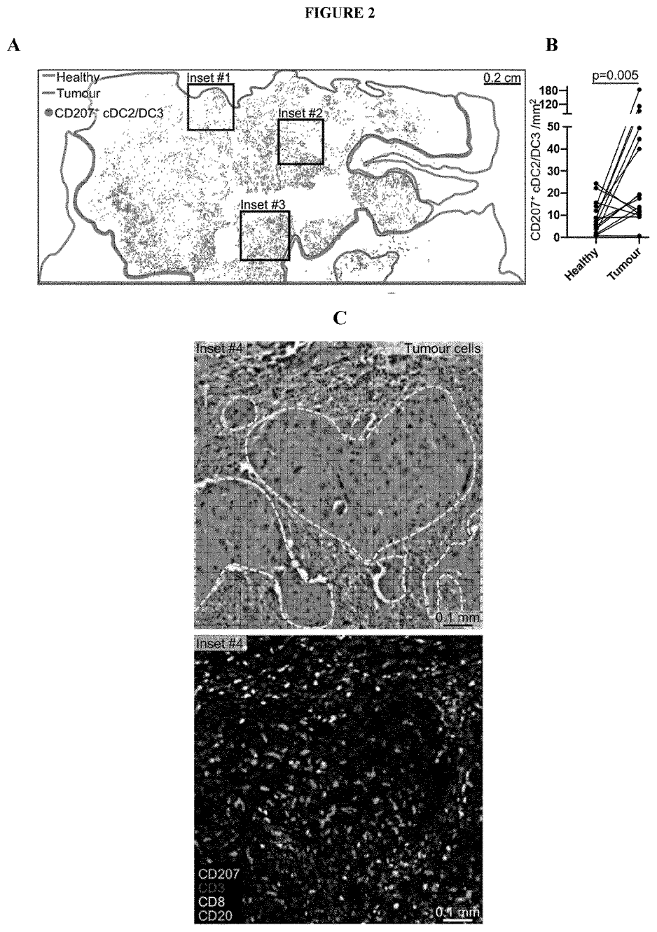

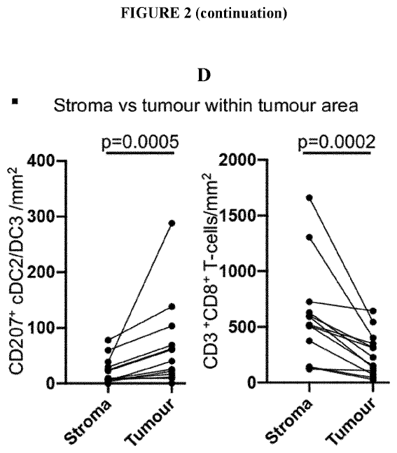

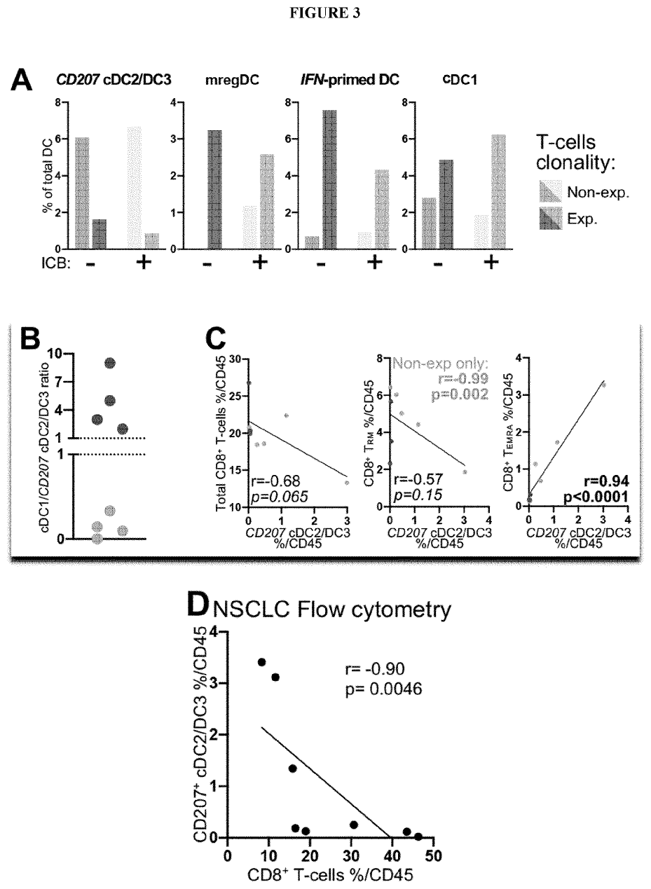

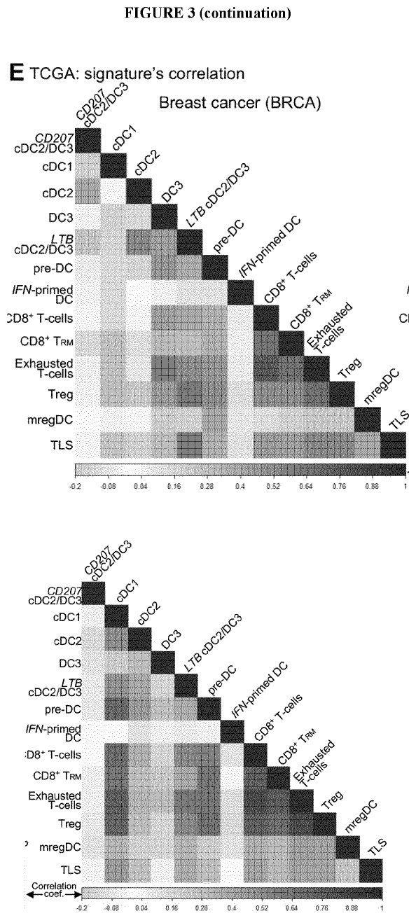

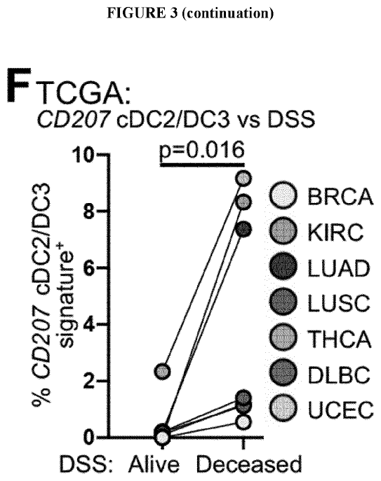

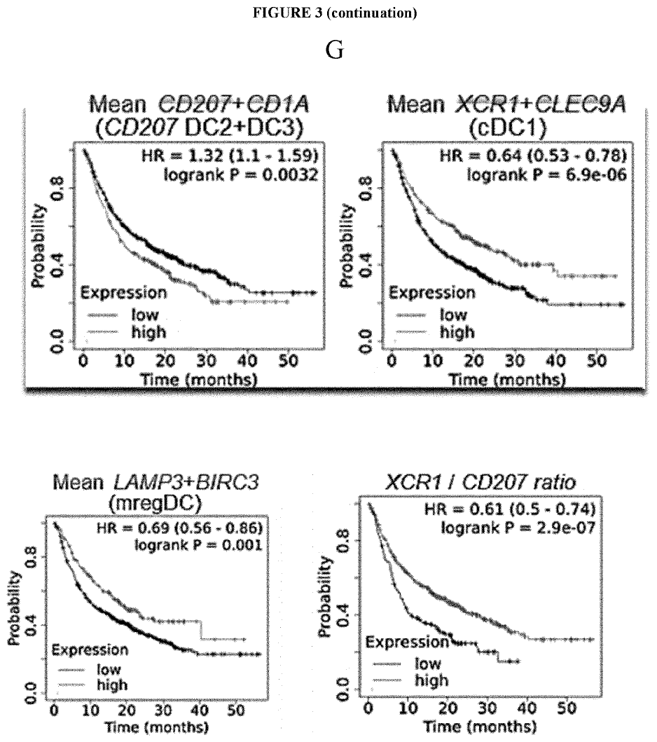

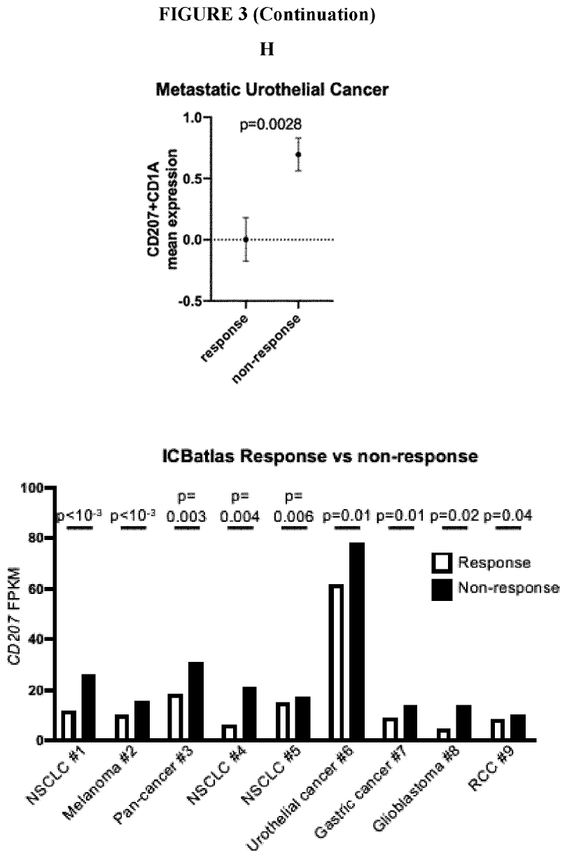

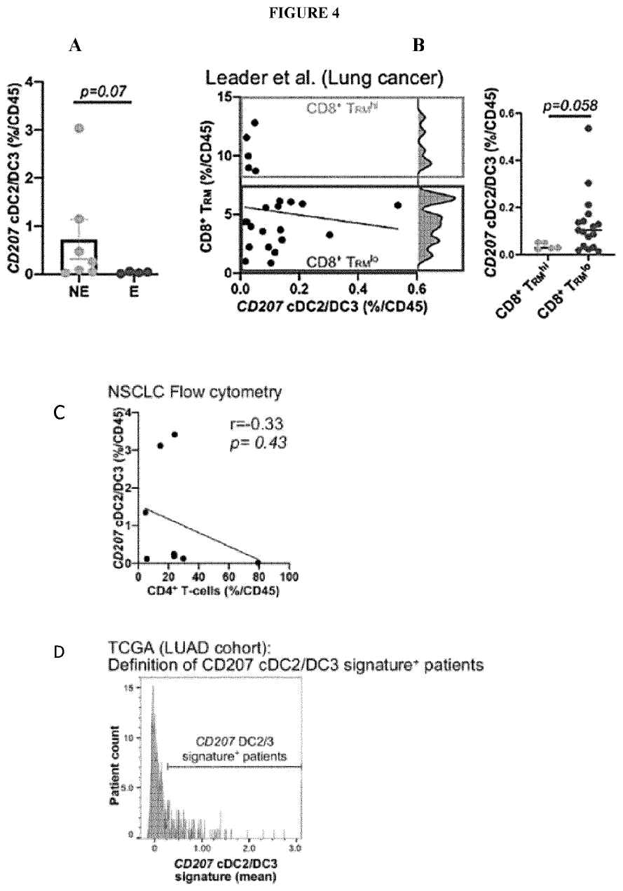

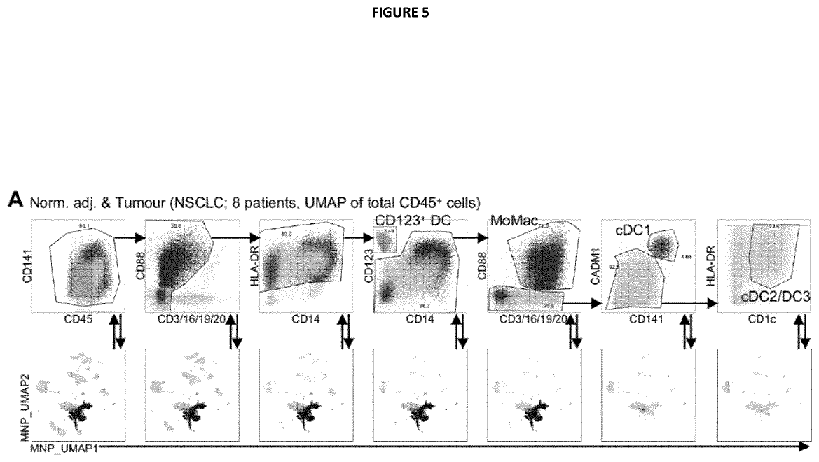

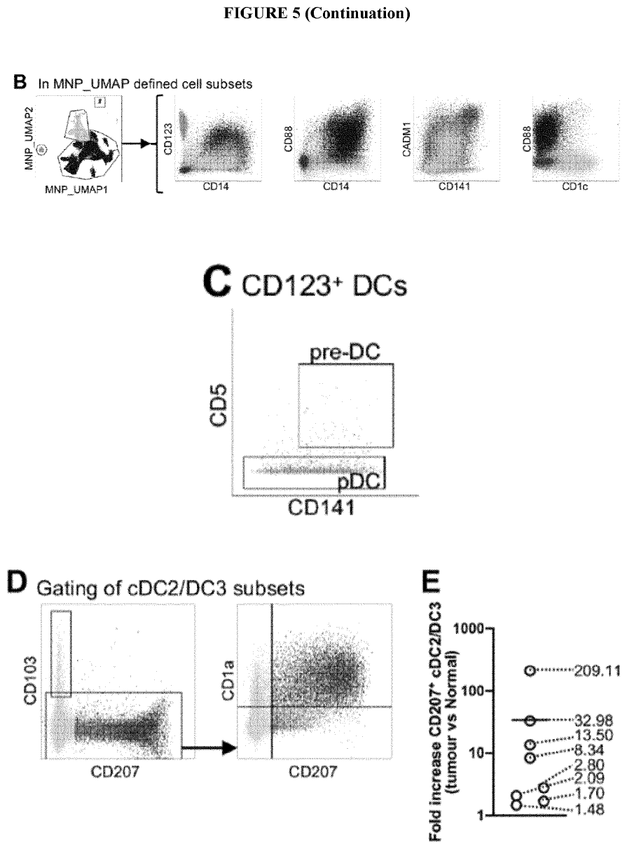

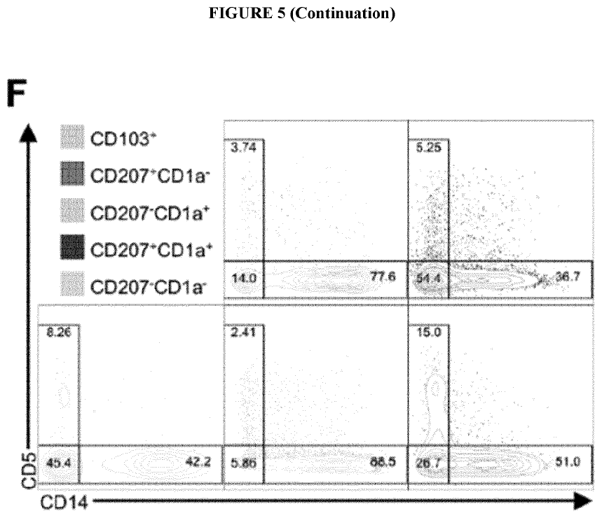

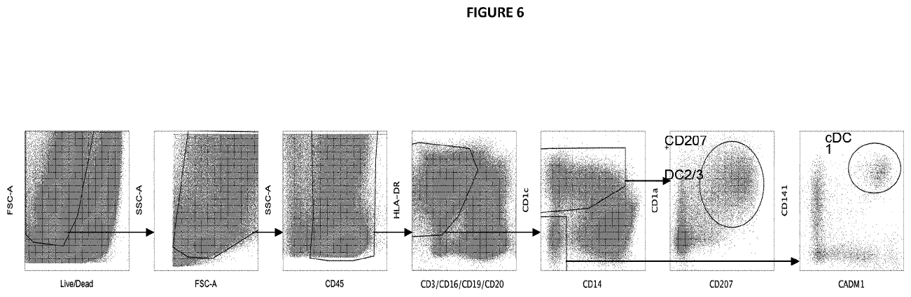

Gating strategy to define DCs and the phenotype of CD207 DC2+DC3 in NSCLC spectral flow cytometry data. (A) Gating strategy from singlets, live, CD45⁺ cells and projection of each gated population onto the MNP_UMAP space. (B) MNP_UMAP annotation confirmed by protein expression. (C) Gating of pDC and pre-DC within CD123⁺ DCs defined in FIG. 1A-B. (D) Gating of CD103⁺ “LTB” and CD1a⁺CD207⁺ DC2+DC3. (E) Fold increase of CD207⁺ DC2+DC3s in tumour versus normal adjacent lungs. (F) Expression of CD5 and CD14 by DC2+DC3 subsets defined in panel (D).

FIG. 6.

8 colour flow cytometry panel for the quantification of CD207⁺ DC2+DC3 and cDC1 in human tumours.

### EXAMPLES

Similar to the previous study investigating monocyte and macrophage heterogeneity (Mulder et al., 2021), the present results provide an integrated analysis of antigen presenting DCs across multiple tissues in various pathologies to build an in-depth overview (DC-VERSE) of human antigen presenting DC heterogeneity. This analysis takes advantage of 40 scRNAseq datasets, along with flow cytometry validation, CITE-seq protein expression data, spatial genomics and histology, and various analytical pipelines to help define a robust characterisation of human DCs.

LegendScreen analysis of human blood and spleen revealed a conserved signature of protein markers to define DC subsets while at the same time demonstrating phenotypic variations across the two tissues. The use of key markers can result in a clear definition of cDC1s, pre-DCs, cDC2s and DC3s from additional MNP populations. Nevertheless, defining DC2+DC3 subsets has proven to be difficult, and several attempts have been made to classify these populations in recent years (Bourdely et al., 2020; Brown et al., 2019; Cheng et al., 2021; Cytlak et al., 2019; Dutertre et al., 2019; Villani et al., 2017). It was previously proposed that CD1c⁺ cDCs (initially called cDC2s) could be broadly divided into CDP/pre-DC-derived cDC2s and CDP/pre-DC-independent DC3s, which include inflammatory CD14⁺DC3s (Dutertre et al., 2019). Using a different nomenclature, Brown et al., recently suggested cDC2A and cDC2B subsets, which we show here resemble Dutertre et al. CD5⁺ cDC2s and CD5 CD14^{+/-} DC3s, respectively, at both protein and RNA levels. While Brown et al., claimed cDC2As were absent from the blood, we could clearly detect blood CLEC10A^{lo}CLEC12A^{lo}CLEC4A^{hi} cDC2A-like cells that also expressed the CD5 protein (based on indexed-FACS data), which is a cDC2 defining marker (Dutertre et al., 2019). Thus, although no TBX21 (T-bet transcript) nor RORC (RORγt transcript) were detected in human blood DC2+DC3 subsets (in bulk RNA-seq data which is much deeper in reads than scRNAseq), the absence of T-bet transcript detection cannot allow to conclude to an absence of cDC2A in human blood. Furthermore, by establishing the DC-VERSE, the present results revealed a greater level of resolution of DC2+DC3 heterogeneity by combining the power of the integration of a large number of cells that revealed discrete cell subsets/states, but also through the combination with protein expression data (CITE-seq and indexed-FACS data). Altogether, this allows to detect that cDC2A_Brown were composed of LTB DC2+DC3s and CD5⁺ cDC2s. The added advantage of the CITE-seq data from Maier et al., (Maier et al., 2020) included in the DC-VERSE allows to show that LTB DC2+DC3s falls within the CD5⁻CD14⁻ fraction of cells, thus validating the observation that half of cDC2A Brown shared this phenotype as observed by mapping cDC2A_Brown on the FACS protein expression data. The separation of cDC2A_Brown in two phenotypically and transcriptionally distinct cell subsets, perfectly overlapped with Brown et al.'s two cDC2A clusters (clusters cl.3 and cl.4) strengthens the present approach that allows, through this large integration, to better resolve DC subsets. In the same line, Cheng et al., also examined DC2+DC3 heterogeneity by scRNAseq (Cheng et al., 2021). For example, the “CD1A DC2s” cluster from Cheng et al., was in fact composed of the LTB and CD207 DC2+DC3 populations which had not been appreciated most probably due to the low abundance of these cells. Altogether, in combination with the Azimuth pipeline, the DC-VERSE can aid researchers in annotating their populations at a much higher resolution.

The increasing number of scRNAseq studies has resulted in various names assigned to DC clusters that share strong similarities, which calls for a nomenclature unification. DC3s were initially proposed as being a subset of CD1c⁺ “DC2s” that harbour pro-inflammatory CD14⁺ cells (Bourdely et al., 2020; Dutertre et al., 2019). Despite these observations, the term “DC3” has also been used to describe cells (Di Pilato et al., 2021; Gerhard et al., 2020; Zilionis et al., 2019), that have an obvious transcriptomic alignment to mregDCs (Di Pilato et al., 2021; Maier et al., 2020; Zheng et al., 2017). Consequently, using the term “DC3” to describe these two fundamentally different populations can be confusing for researchers (Ginhoux et al., 2022). In an attempt to resolve this, the present inventors showed here how published signatures aligned to the DC populations identified here in the DC-VERSE. For example, the various nomenclature assigned to mregDCs included migratory/activated/LAMP3/CCR7/“DC3”. Since the DC-VERSE considers several datasets across various diseases and tissues, the present analysis is one of the most in-depth and comprehensive analyses of antigen presenting DC heterogeneity. Therefore, to avoid any further confusion, it is proposed to use in the future the nomenclature used here.

The DC-VERSE allowed to investigate DC2+DC3 heterogeneity and profile antigen presenting DCs across steady state tissues and disease. By integrating a large number of cells, the DC-VERSE also allowed to automatically exclude/annotate senescent cells (that fell within the Low_nFeat. cl. #20), although lower quality cells were already excluded prior to integration. Another advantage of this approach was that focusing solely on DCs allowed reducing the data's global variance, which revealed discrete cell subsets during dimensionality reduction (PCA within the Seurat V3 pipeline followed by UMAP and unsupervised clustering).

The present inventors particularly focused on the changes of DC heterogeneity between healthy and tumour tissues. By doing so, they observed genes such as SPP1, which were highly expressed in tumour tissues mirroring findings also observed in monocytes and macrophages (Mulder et al., 2021), but also revealed a downregulation of genes such as the cDC1-specific CLEC9A gene that already pointed out to a reduction of cDC1s' density within human tumours (Kvedaraite and Ginhoux, 2022). Recently, an enrichment of “ISG⁺ DC2s” was observed in the tumours of mice, which efficiently activate CD8⁺ T-cells, thus promoting anti-tumour immunity (Duong et al., 2021). The present inventors also identified IFN-primed DCs in human tumours, which were also increased in the majority of cancers compared to normal adjacent tissue. Additionally, the expansion of mregDCs and DC3s has been reported in lung cancer patients (Leader et al., 2021; Maier et al., 2020), which was extended to several cancers. They further confirmed this by flow cytometry in 7 out of 8 NCSLC patients. Of note, while Leader et al., observed an expression of CD1A and CD207 transcripts by tumour DC2+DC3s, the inventors were able to clearly delineate a discrete subset of CD207⁺CD1A⁺ DC2+DC3s (CD207 DC2+DC3s). This population was of significant interest as its expansion was observed in all cancers included in the DC-VERSE with further validation by spectral flow cytometry in all eight NSCLC patients analysed. The spectral flow cytometry analysis also suggested that the CD103⁺ “LTB” and CD207⁺ CD1a⁺ DC2+DC3 populations could correspond to an activation “state” rather than an ontogenically distinct cell subsets since they both exhibit DC2 (CD5⁺) and DC3 (CD5⁻CD14^{+/-}) phenotypes. This is similar to mregDCs, which correspond to a mature (potentially migratory) “state” and not to a distinct DC subset. Indeed, mregDCs comprise both cDC1s, DC2 and maybe DC3s that have engaged a common maturation gene expression program (Maier et al., 2020).

### MATERIAL AND METHODS

### Human Tissue and Blood Samples

Tumour and healthy adjacent tissue from the lung as well as FFPE blocks, were obtained from Non-Small Cell Lung Cancer patients following written informed consent (Marie Lannelongue Hospital, Paris) and ethical approval (N°ID-RCB: 2016-A00732-49). All subjects provided IRB-approved consent. Human tissues were cut into 0.5 cm squares and incubated with 0.8 mg/mL collagenase (Type IV, Worthington-Biochemical) in RPMI (PAA) with 10% FCS (AutogenBioclear) for 2 and 8 h, respectively, or when stated mechanically dispersed.

### LegendScreen and InfinityFlow Pipeline

The analysis of the Legendscreen and InfinityFlow pipelines are based on the methods previously described in Dutertre et al., 2019.

Separate LegendScreen experiments (using two versions of the LegendScreen kit that did not contain the exact same PE-conjugated antibodies) were performed on each tissue using the following protocol. PBMCs and splenic mononuclear cells were incubated with Live/Dead blue dye (Invitrogen) for 30 min at 4 °C in phosphate buffered saline (PBS) and then incubated in 5% heat-inactivated fetal calf serum (FCS) for 15 min at 4 °C (Sigma Aldrich). The following 14 anti-backbone markers antibodies were added to the cells and incubated for 30 min at 4 °C, and then washed: CD123-BUV395 (clone 7G3), HLA-DR-BV786 (clone L243), CD5-BV711 (clone UCHT2), CD3-BV650 (clone SP34-2), CD20-BV650 (clone 2H7), CD45-V500 (clone HI30), CD2-BV421 (clone RPA-2.10), CD45RA-FITC (clone 5H9), CD14-AlexaFluor700 (clone M5E2), all from BD Biosciences; CD163-BV605 (clone GHI/61), CD1c-PercP/Cy5.5 (clone L161), CD88-PE/Cy7 (clone S5/1), CD16-APC/Cy7 (clone 3G8), all from Biolegend; CD141-APC (clone AD5-14H12, Miltenyi Biotec). The cells were then stained with 332 or 361 different PE-conjugated antibodies for blood and spleen, respectively, using the LegendScreen Human PE kit (Biolegend) following the manufacturer's instructions. Subsequent LegendScreen analysis of human blood and spleen mononuclear phagocytes was carried out using the InfinityFlow pipeline as previously described in Dutertre et al. (Dutertre et al., 2019). This involves the regression analysis of the intensities of the PE-bound markers using the intensities of the backbone markers. In detail, the compensated cytometry data were transformed using a logicle transformation with parameters w=0.1, t=500000, m=4.5 and a=0, as defined in the flowCore R package. Half of the events were randomly selected for each FCS file of the Legend Screen (Biolegend) experiment to train an epsilon-regression Support Vector Machine (SVM) model using the e1071 R package with default parameters, resulting in 332 or 361 SVM regression models. For each model, the PE-bound marker intensity was used as the response variable, and the intensities of the backbone markers were used as independent variables. Each SVM regression model was applied on its associated vector of backbone marker intensities to predict the intensities of 332 or 361 PE-bound markers for each event. For each of the 332 or 361 initial Legend Screen fcs files, these 332 or 361 regressed values were transformed to a linear intensity scale, concatenated with the backbone and the PE-marker expression values exported back as 332 or 361 new single .fcs files. These predictions were used as the input for t-distributed Stochastic Neighbour Embedding (t-SNE) dimensionality reduction (using the Barnes-Hut implementation of t-SNE from the Rtsne R package) and Phenograph clustering from the Rphenograph R package. Heatmap showing the z-score of discriminating markers were plotted using hierarchical clustering by Euclidean distance in Prism 8.

### Flow Cytometry

Cells were thawed from liquid nitrogen and transferred into RPMI (ThermoFischer) with 20% decomplemented FCS (ThermoFischer). Samples were treated with 1 mg/ml DNase I (Sigma-Aldrich) at 37 °C. Cells were incubated with Live/Dead blue dye (Invitrogen) for 30 min at 4 °C in phosphate buffered saline (PBS) and then incubated in 5% heat-inactivated fetal calf serum (FCS) (Sigma Aldrich) for 15 min at 4 °C. Cells were stained with appropriate antibodies in PBS with 2% FCS and 2 mM EDTA (Sigma Aldrich) and Brilliant Stain buffer (BD) and incubated for 30 min at 4 °C, and then washed. Cells were analysed using a Cytek Aurora 5-laser spectral analyser. Fcs files were exported and analysed using FlowJo v10.5.3.

### Immunohisto Fluorescence Labelling Assays

All immunostainings were performed on 4 μm thick whole sections prepared from FFPE blocks of human NSCLC (Marie Lannelongue Hospital, Paris) were used. Antigen retrieval was carried out on a PT-link (Dako) using the EnVision FLEX Target Retrieval Solutions at High pH (Dako, K8004) or Low pH (Dako, K8005). Endogenous peroxidase activity and non-specific Fc receptor binding were blocked with H₂O₂ 3% (Gilbert, 3518646067907) and Protein Block (Dako, X0909) respectively. The primary and secondary antibodies used for immunofluorescence are summarized in the key resources table. Necrotic, serous, folded and blurred areas were excluded from image analyses. The CD20/CD3/CD207/CD8 4-plex staining was performed manually using the OPAL tyramide system amplification (TSA) (Akoya Biosciences). Nuclei were stained with DAPI Solution (Thermofisher, 62248) at 2 μg/ml for 5 minutes. After mounting with ProLong™ Glass Antifade Mountant (Thermofisher, P36980), the slides were scanned at 20× magnification using a Zeiss Axio scan Z1 device. Antibodies and TSA used are listed in this table:

| | | |
|---|---|---|
| REAGENT or RESOURCE | SOURCE | IDENTIFIER |

Antibodies for spectral flow cytometry

| | | |
|---|---|---|
| Anti-goat (Polyclonal) AF488 | Jackson Immuno- | Cat# 705-545-147, RRID: AB_2336933 |
| | Research | |
| | Laboratory | |
| CCR2 (Clone LS132.1D9) BUV563 | BD Biosciences | Cat# 749076, RRID: AB_2873470 |
| CD11b (Clone M1/70) BV570 | Biolegend | Cat# 101233, RRID: AB_10896949 |
| CD117 (Clone 104D2) APC | Biolegend | Cat# 313206, RRID: AB_314985 |
| CD123 (Clone 6H6) PE/Daz594 | Biolegend | Cat# 306034, RRID: AB_2566450 |
| CD103 (Clone Ber-ACT8) BV605 | Biolegend | Cat# 350217, RRID: AB_2564282 |
| CD14 (Clone 63D3) SparkB550 | Biolegend | Cat# 367148, RRID: AB_2832724 |
| CD141 (Clone M81) BV421 | Biolegend | Cat# 344114, RRID: AB_2562956 |
| CD169 (Clone 7-239) BUV661 | BD Biosciences | Cat# 750363, RRID: AB_2874538 |
| CD19 (Clone HIB19) BV650 | Biolegend | Cat# 302238, RRID: AB_2562097 |
| CD1a (Clone SK9) BB660 | BD Biosciences | Custom |
| CD1c (Clone L161) SB436 | Invitrogen | Cat# 62-0015-42, RRID: AB_2762426 |
| CD20 (Clone 2H7) BV650 | Biolegend | Cat# 302336, RRID: AB_2563806 |
| CD25 (Clone CD25-3G10) | Thermo Fisher | Cat# MHCD2524, RRID: AB_2539740 |
| PE/AF700 | Scientific | |
| CD206 (Clone 19.2) PercP/eFluor | Thermo Fisher | Cat# 46-2069-42, RRID: AB_10853034 |
| 710 | Scientific | |
| CD207 (Clone 2G3) BB755 | BD Biosciences | Custom |
| CD209 (Clone 9E9A8) PE/Cy7 | Biolegend | Cat# 330114, RRID: AB_10719953 |
| CD3 (Clone SP34-2) BV650 | BD Biosciences | Cat# 563916, RRID: AB_2738486 |
| CD301 (Clone H037G3) PE | Biolegend | Cat# 354704, RRID: AB_11219002 |
| CD38 (Clone HB7) BUV805 | BD Biosciences | Cat# 742074, RRID: AB_2871359 |
| CD4 (Clone SK3) Cfl.YG584 | Cytek Biosciences | Cat# R7-20041, RRID: AB_2885083 |
| CD45 (Clone 30-F11) PercP | Biolegend | Cat# 103130, RRID: AB_893339 |
| CD5 (Clone UCHT2) APC/R700 | BD Biosciences | Cat# 565121, RRID: AB_2744433 |
| CD8 (Clone SK1) APC/Fire 810 | Biolegend | Cat# 344764, RRID: AB_2860890 |
| CD127 (Clone HIL-7R-M21) BB510 | BD Biosciences | Custom |
| CD86 (Clone 2331) BV786 | BD Biosciences | Cat# 740990, RRID: AB_2870657 |
| CD88 (Clone S5/1) APC/Fire750 | Biolegend | Cat# 344316, RRID: AB_2750445 |
| CD89 (Clone A59) BUV496 | BD Biosciences | Cat# 750617, RRID: AB_2874749 |
| CD9 (Clone M-L13) BV480 | BD Biosciences | Cat# 746356, RRID: AB_2743675 |
| FcεRlα (Clone AER-37 (CRA-1)) | Biolegend | Cat# 334638, RRID: AB_2687186 |
| BV711 | | |
| HLA-ABC (Clone W6/32) Sp. NIR | Biolegend | Cat# 311453, RRID: AB_2876612 |
| 685 | | |
| HLA-DP (Clone B7/21) BUV395 | BD Biosciences | Cat# 750866, RRID: AB_2874962 |
| HLA-DQ (Clone Tu169) BV510 | BD Biosciences | Cat# 742609, RRID: AB_2740907 |
| HLA-DR (Clone L243) PE/Fire 810 | Biolegend | Custom |
| PD-L1 (Clone MIH1) PE/Cy5 | Invitrogen | Cat# 15-5983-42, RRID: AB_2802211 |
| PD1 (Clone EH12.1) BV750 | BD Biosciences | Cat# 747446, RRID: AB_2872125 |
| SLAN (Clone DD-1) VioBlue | Miltenyi Biotec | Cat# 130-119-868, RRID: AB_2733608 |
| CADM1 (Clone 3E1) | MBL International | Cat# CM004-3, RRID: AB_592783 |
| LIVE/DEA ™ Fixable Blue Dead | Invitrogen | Cat# L23105 |
| Cell | | |
| CCR7 (Clone 2-L1-A) BUV737 | BD Biosciences | Cat# 749676, RRID: AB_2873937 |

Antibodies and reagents for immunohisto fluorescence experiments

| | | |
|---|---|---|
| Rabbit IgG polyclonal anti-CD3 | Agilent | Cat#: A0452, RRID: AB_2335677 |
| (6 μg/mL) | | |
| Mouse IgG1 mAb (C8/144B) | Agilent | Cat#: M7103, RRID: AB_2075537 |
| anti-CD8 (1.6 μg/mL) | | |
| Mouse IgG2a mAb (L26) anti-CD20 | Agilent | Cat#: M0755, RRID: AB_2282030 |
| concentrated at 0.3 μg/mL | | |
| Rat IgG2a mAb (929F3.01) | Eurobio Scientific | Cat#: DDX0362P-100, RRID: AB_2892751 |
| anti-CD207/Langerin (2.5 μg/mL) | | |
| Polymer anti-Mouse HRP | Agilent | Cat#: K4001, RRID: AB_2827819 |
| Polyview ® plus anti-Rabbit HRP | Enzo Life | Cat#: ENZ-ACC103-0150 |
| | Sciences | |
| Opal Polymer anti-Rabbit HRP | Akoya | Cat#: ARR1001KT |
| | Biosciences | |
| Polymer IgG anti-Rat HRP | Vector | Cat#: MP-7404, RRID: AB_2336531 |
| | Laboratories | |
| OPAL 520 | Akoya | Cat#: FP1487001KT |
| | Biosciences | |
| OPAL 570 | Akoya | Cat#: FP1488001KT |
| | Biosciences | |
| OPAL 690 | Akoya | Cat#: FP1497001KT |
| | Biosciences | |
| OPAL Polaris 780 | Akoya | Cat#: FP1501001KT |
| | Biosciences | |

Chemicals, peptides, and recombinant proteins for immunohisto fluorescence experiments

| | | |
|---|---|---|
| EnVision FLEX Target Retrieval | Agilent | Cat#: K8004 |
| Solution, High pH | | |
| EnVision FLEX Target Retrieval | Agilent | Cat#: K8005 |
| Solution, Low pH | | |
| H2O2 3% | Gilbert | Cat#: 3518646067907 |
| Protein Block | Agilent | Cat#: X0909 |
| DAPI | Thermofisher | Cat#: 62248 |
| ProLong ™ Glass Antifade Mountant | Thermofisher | Cat#: P36980 |

Chemicals, Peptides, and Recombinant Proteins

| | | |
|---|---|---|
| DNAse I | Sigma Aldrich | Cat# 48024000 |
| PBS | ThermoFisher | Cat# 20012-027 |
| Collagenase IV | Sigma Aldrich | Cat# C5138-500MG |
| RPMI | ThermoFisher | Cat# 31870-025 |
| Brilliant Stain Buffer | BD Biosciences | Cat# 563794 |
| FCS | ThermoFisher | Cat# 26140079 |

Biological samples

| | | |
|---|---|---|
| Matched LUAD lung sample | Marie | Matched LUAD lung sample |
| | Lannelongue | |
| | Hospital, Paris | |

Software and Algorithms

| | | |
|---|---|---|
| Anaconda 2018.12 | Anaconda | https://www.anaconda.com |
| DIVA | BD Biosciences | https://www.bdbiosciences.com/en-us |
| FlowJo v10.5.3 | Tree Star | https://www.flowjo.com |
| GraphPad Prism 6 | GraphPad | https://www.graphpad.com/scientificsof |
| | | tware/prism/ |
| Ingenuity Pathway Analysis v01-16 | QIAGEN | https://www.giagenbioinformatics.com/ |
| | | products/ingenuity-pathway-analysis/ |
| NicheNet | Browaeys et al., | https://github.com/saeyslab/nichenetr |
| | 2019 | |
| Phenograph | Levine et al., 2015 | https://github.com/JinmiaoChenLab/Rp |
| | | henograph |
| Python v3.7.1 | Python Software | https://www.python.org |
| | Foundation | |
| R v4.4 | The R Foundation | https://www.r-project.org |
| SCENIC | Van de Sande | https://github.com/aertslab/pySCENIC |
| | et al., 2020 | |
| SeqGeq v1.6 | FlowJo | https://www.flowjo.com/solutions/seqgeq |
| Seurat v3 | Stuart et al., 2019 | https://satijalab.org/seurat/ |
| Seurat v4 | Hao et al., 2020 | https://satijalab.org/seurat/ |
| UMAP | Mclnnes et al., | https://github.com/Imcinnes/umap |
| | 2018 | |
| Halo version 3.4.2986.166 | Indica Labs | RRID: SCR_018350 |
| | | https://indicalab.com |

### Algorithms for Dimensionality Reduction

For flow cytometry data, marker expression values were transformed using the auto-logicle transformation function from the flowCore R package. Uniform Manifold Approximation and Projection (UMAP) were carried out using all markers (flow cytometry) or significant PCs (based on Seurat analysis for scRNAseq data). UMAP was run using 15 nearest neighbours (nn), a min_dist of 0.01 to 0.2 and Euclidean distance (Becht et al., 2018; McInnes et al., 2018). Phenograph clustering (Levine et al., 2015) was performed using all markers or significant PCs (based on Seurat analysis) before dimension reduction. The number of PCs selected was equal to 50 for the DC-VERSE, 30 for flow cytometry and equal to 15 for scRNAseq analysis.

### MNP Extraction and Seurat V3 Integration

41 datasets examining monocyte and macrophage heterogeneity were previously integrated (Mulder et al., 2021). Similarly, for DCs we integrated 40 of the 41 datasets (1 of the 41 datasets did not meet the cell number criteria for integration). The 40 datasets used were either at the raw count matrix or already pre-processed and filtered. As previously described (Mulder et al.), all the datasets were first integrated in an organ-specific manner. Before integrating the datasets, universal quality control was applied to keep everything in a unified matter. Cells that expressed fewer than 500 genes or had more than 20% mitochondrial reads were filtered out. All datasets were then unified in the same expression matrix format. Integration was initiated using the Seurat V3 anchoring method (Stuart et al., 2019) and log normalised. The matrix was scaled, and a Principal Component Analysis (PCA) was performed (Becht et al., 2018), from which the first 50 significant Principal Components (PCs) were selected for UMAP analysis. Following the identification of MNPs using canonical markers, a global integration (using 50 PCs for dimensional reduction) of dendritic cells from all tissues was carried out as above.

### Quantification and Statistical Analysis

### Differentially Expressed Genes (DEGs)

DEG analyses were performed using the Seurat v3 package (Stuart et al., 2019). DEGs obtained from the “RNA” matrix of the Seurat object were calculated on normalised values with a log FC threshold of 0.25, and the threshold for false discovery is 0.05. The likelihood-ratio test for single-cell gene expression (bimodal test) was used, and correction for multiple testing was carried out using the Bonferroni method. For the generation of the DEG heatmap, pairwise DEGs were calculated for all possible combinations between LCH, LC and CD207 DC2+DC3. Uniquely expressed DEGs and shared DEGs between populations were plotted using the Seurat heatmap function.

### Dendrogram Heatmap to Define DC-VERSE Mega Clusters

The average expression of each gene obtained from the DEG analysis for each Phenograph cluster was first obtained, and a Spearman correlation was calculated. Dendrogram heatmap was then generated using pHeatmap using the Ward's method for hierarchical cluster analysis.

### Published DC Signature Score Heatmap

For the generation of the signature score heatmap, the package pHeatmap was used as described above. The signatures were obtained from various public datasets. The DC-VERSE was split into the defined mega clusters in which we obtained the average gene expression of every mega cluster for all the genes present. Following this, the mean expression of the signature is calculated and plotted by the mega clusters in the heatmap.

### Metadata Analysis

Metadata analysis was performed for selected studies with paired conditions (healthy versus cancer). The proportion of mega clusters was plotted for each state as charts and density plots for the selected studies. Further analysis was performed to deconvolute at the patient level in datasets where this information was provided. We only analysed datasets where more than 7 cells were present. Charts and density plots were made in GraphPad Prism v6 and SeqGeq v1.6, respectively. Statistical tests were performed using GraphPad Prism v6 and are specified within the figure legends.

### Scenic Gene Regulatory Network Analyses

To infer gene regulatory networks (GRNs) from tpm-normalized expression matrices of Lung (Maier et al., 2020) and Head/Neck (Cillo et al., 2020), a pySCENIC (single-cell regulatory network inference and clustering) v0.10.3 analysis was performed (Van de Sande et al., 2020).

The analysis consisted of three main steps (GitHub/pySCENIC): generation of co-expression modules with GRNBoost2, refinement of these modules with RcisTarget and evaluation of the regulon activity with AUCell (Van de Sande et al., 2020). Differentially expressed regulons (DERs) were calculated using the Seurat pipeline with the same parameters as described above for DEGs analysis (adjusted p-value lower or equal to 0.05 and Log 2FC cut-off of 0.25). Phenograph cluster-specific DERs and DERs that had similar expression patterns across closely related Phenograph clusters were identified and subsequently used to generate a heatmap (not shown).

### Spatial Transcriptomic Analysis (Visium 10× Genomics)

Analysis are performed as previously described in the paper of Wu et al. (Wu et al., 2021).

Spatial transcriptomic data for Visium samples were deconvoluted using Stereoscope 0.3.1 using Wu et al. Breast cancer scRNAseq reference (Wu et al., 2021). Cells in the scRNAseq reference were filtered with the—sc_upper_bound option so that no more than 1000 (randomly selected) cells of any given cell type were used to fit the model. Similarly, genes were filtered by a list of 3695 highly variable genes, as identified using the sc.pp.highly_variable_genes( ) function from Scanpy with default parameters and the—filter-genes option was also enabled. Both model fitting and model application was run for 75000 epochs each, with a batch size of 100. Wu et al. pathology annotations were used to calculate enrichment of spatially deconvoluted cell subsets, and deconvoluted proportion results were mapped onto H&E images using Seurat v4.

### Pathway Analysis

DEGs of DC populations, together with the respective fold-change and p-values, were uploaded to the Ingenuity Pathway Analysis (IPA) software (QIAGEN). IPA analysis reported the p-value of canonical pathways. Predicted upregulated or downregulated pathways were represented by a positive or negative Z-score, respectively. Canonical pathways are determined by IPA's default threshold [−log. (p-value)>1.3] were then shortlisted, and bubble plots were used to visualize the p-values and Z-scores.

### TCGA Analysis

For the generation of the correlation matrix, signatures were used from Mulder et al., Nalio Ramos et al. and Mackay et al. (Mackay et al., 2016; Mulder et al., 2021; Nalio Ramos et al., 2022). The TCGA datasets were scaled by Z-score per patient, of which the average of every signature was calculated. A Pearson correlation was used to obtain the correlation matrix and plotted using the corrplot R package (0.84).

### Quantification and Statistical Analysis of the Immunohisto Fluorescence Labelling

Tumour and normal adjacent tissue areas were defined by a pathologist (G.G.). Within tumour, tumour nests and stromal areas were identified using the Halo10 software (Indica labs) with a classifier based on examples of the two areas. The density of positive cells/mm²: CD3⁺, CD8⁺, CD20⁺, and CD207⁺ cells were quantified in the different zones with Halo10 software (Indica labs) using the fitting counting algorithms.

### Results

### DC-Verse

The inventors recently performed a meta-analysis of human macrophages and monocytes sequenced from 41 published datasets across 13 tissues in health and disease (Mulder et al., 2021). They adopted a similar approach for cDCs, where datasets were first integrated tissue by tissue using the Seurat V3 pipeline, from which cDCs were then extracted and re-integrated into a UMAP space to generate the DC-VERSE (not shown). They used the Phenograph algorithm (Levine et al., 2015) to define clusters and calculated the DEGs and differentially expressed regulons (DERs) for all Phenograph clusters (cl. #) using the RNA matrix (not shown). Cl. #20 consisted of cells with a relatively low nfeature_RNA (not shown) and cl. #15 was a minor population of 118 cells that were consequently not examined further (not shown).

The inventors examined the expression of well-established signature genes and proteins (using CITE-seq data provided by Maier et al.) to broadly identify major DC subsets (not shown). TCF4/AXL transcripts and CD45RA/CD123 protein expression identified pre-DCs; CADM1/CLEC9A transcripts and CD141/CD26 proteins identified cDC1s; FCER1A/CD1C transcripts and CD33/CD1c proteins identified DC2+DC3s; BIRC3/LAMP3 transcripts and PD-L1/PD-L2 proteins identified mregDCs and PCNA/MKI67 transcripts identified proliferating cDCs (Prolif.). In addition, they observed that cl. #11 cells expressed T-cell specific membrane proteins, which likely represent T/DC cell doublets (not shown). They next implemented dendrogram clustering to understand the relationship of Phenograph clusters to one another and recognised twelve DC mega-clusters (not shown). In particular, they identified regions encompassing CD207 DC2+DC3s (cl. #7), LTB DC2+DC3s (cl. #3 and #5), pre-DCs (cl. #19), cDC1s (cl. #8 and #10), cDC2s (cl. #12, #13 and #17), DC3s (cl. #1, #4 and #9), mregDCs (cl. #14 & #16), IL1B DC2+DC3s (cl. #18), proliferating DCs (Prolif.) (cl. #2 and #6) and T/DC doublets (cl. #11) [low nfeature (low nfeat.) cells (cl. #20) and a minor (cl. #15) were not analysed further as mentioned above]. Although cl. #17 was grouped as related to cDC2s, given the unique gene expression of IFN-related genes, they were considered as an independent mega-cluster that we called IFN-primed DCs (not shown). Dendrogram clustering helped to define cDC2 and DC3 mega-clusters, alongside cDC2 and DC3 proteins from Maier et al.'s CITE-seq data (Maier et al., 2020), and cDC2 and DC3 gene signatures from Dutertre et al. (Dutertre et al., 2019) (not shown). Given the close nature of cDC2s and DC3s, they further used CD5 and CD14 from CITE-seq data (Maier et al., 2020) to identify CD5⁺ cDC2s and CD14⁺ DC3s, which were then backgated onto the DC-VERSE to visualise their location within (not shown). They also highlighted cl.3 and cl.4 splenic cDC2A Brown on the DC-VERSE, revealing that cl.3 corresponds to LTB DC2+DC3s while cells from cl.4 were detected within CD5⁺ cDC2s (not shown).

### The DC-Verse Establishes Conserved DC Subsets and States Across Human Tissues in Health and Disease

The inventors subsequently performed DEG analysis across mega-clusters to establish signature genes for each of these populations (not shown). In addition to finding signature genes for widely recognised DC subsets (mregDCs, pre-DCs, cDC2s, DC3s and cDC1s), they also identified genes for less characterised/well-known DC subsets such as LTB and LST1 for LTB DC2+DC3s; CD1A, CD207 and HLA-DQB2 for CD207DC2+DC3s; ISG15, IFI6, IFI44L and IFIT3 for IFN-primed DCs and finally AREG, IL1B, CCL17 and NFKB1 for IL1B DC2+DC3s. DC subsets and states were detected in varying frequencies across healthy tissues, with some clusters such as mregDCs, LTB and CD207 DC2+DC3s detected only in tissues but not in blood (not shown).

Of note, DC2+DC3 heterogeneity in humans has also been recognised by Cheng et al. (Cheng et al., 2021). In this study, the authors performed an in-depth analysis of DC2+DC3 subsets in various human cancer settings, describing six DC2+DC3 subsets (Cheng et al., 2021). As these data were not integrated with the initial 40 datasets at the time, the inventors took advantage of the Azimuth tool, which allows the projection of a query dataset onto a reference map (Hao et al., 2021). After projecting the DC2+DC3 subsets onto the DC-VERSE, they could observe which mega-clusters these cells were assigned to (not shown). Interestingly, the DC-VERSE could filter out cells with a low nFeature_RNA as they mapped to the DC-VERSE cl. #20 (not shown). When examining the mean expression of the top 50 DEGs of all DC2+DC3 DC-VERSE mega-clusters on the UMAP from Cheng et al., they observed how the populations identified by these authors corresponded to the DC-VERSE mega-clusters (not shown) and vice versa, observing their signatures on the DC-VERSE (not shown). The cDC2 ISG15 cluster defined by Cheng et al., corresponded to the IFN-primed DC3s and the cDC2_FCN1 cluster defined by Cheng et al., corresponded to DC3s. In turn, cells with the signature of LTB DC2+DC3s and of CD207 DC2+DC3s (both defined within the DC-VERSE) corresponded to unique subsets of Cheng et al.'s cDC2_CD1A (not shown), highlighting the strength of the DC-VERSE to identify small discrete populations which might otherwise be missed.

After performing DEG analysis of the DC-VERSE mega-clusters, the inventors performed a gene regulatory network analysis (SCENIC) to identify mega-cluster-specific regulons common to two datasets [Lung (Maier) and Tonsil (Cillo)] (Cillo et al., 2020; Maier et al., 2020), which adequately represented the UMAP space (not shown). Regulons included SOX4 and KLF3 for pre-DCs, IRF8, KLF8, FOXB1, HOXA7 for cDC1s, RXRA and RUNX3 for CD207 DC2+DC3s and ZEBI, IRF4, RELB for mregDCs.

To gain insights into the biological processes and functional relevance of these cells, they performed pathway analysis for major DC mega-clusters (not shown). They identified pathways including “Integrin and mTOR signalling” for LTB DC2+DC3s; “IL-10 signalling” and “IL-6 signalling” for IL1B DC2+DC3s, “Aryl Hydrocarbon Receptor (AHR) signalling” and “Dendritic Cell Maturation” for mregDCs, “Inflammasome pathway and complement system” for DC3s, and “ERK/MAPK and HIF1α signalling” for CD207 DC2+DC3s.

Given the vast number of recent publications examining DC subsets, various names have been assigned to DC subsets due to a lack of unity between publications and have consequently created confusion within the field (Ginhoux et al., 2022, 2022). For example, the denomination ‘DC3’ was initially used to describe a subset of CD14⁺ inflammatory DCs, whilst others have used ‘DC3’ to describe DC that have an obvious transcriptomic alignment to mregDCs (Maier et al., 2020), because of the shared expression of transcripts such as BIRC3, CCR7, FSCN1 and LAMP3 among others. Therefore, the inventors sought to align the subsets identified here in the DC-VERSE with that of the published DEG signatures (not shown). The heatmap allowed to unify the multiple identities given to DCs and demonstrated that mature ‘DC3s’ defined by Gerhard et al., and Zilionis et al. (Gerhard et al., 2020; Zilionis et al., 2019), LAMP3 DCs or CCR7 DCs identified by Zhang et al. and Qian et al., respectively (Qian et al., 2020; Zhang et al., 2019) all shared the gene expression signature of mregDCs defined here. Whilst the DC3 signatures [DC3 as defined by Dutertre et al. (Dutertre et al., 2019)] could be appreciated, some of the published signatures are only weakly associated with cDC2s or DC3s.

Altogether, the integrative approach used to generate the DC-VERSE allowed them to appreciate the heterogeneity of cDCs and define universal signatures of their subsets/states, thus offering the possibility to reconcile the different studies that focused on the single-cell transcriptomic analysis of cDCs. They next set out to further unravel the heterogeneity of cDC2s and DC3s and study their potential association with pathology.

### The DC-VERSE Reveals Changes in DC Distribution Across Healthy and Cancer Tissues

To examine the significance of the DC subsets identified here, the inventors performed a metadata analysis examining matched healthy and cancer tissues. They first compared DEGs between matched healthy and cancer tissue across all cDCs (not shown). To this end, they observed DEGs including SPP1, CD14, LTB, CD1A, BIRC3 and CD207 that were more highly expressed in the cancer setting, whereas CLEC9A (a cDC1 specific gene) and CPVL were found to be enriched in the healthy tissues. As several of the DEGs corresponded to the DC-VERSE mega-cluster DEGs, they set out to examine how their relative proportions change across matched healthy and cancer tissues. They first globally compared the proportion of mega-clusters across healthy and cancer matched tissues, where they observed a potential increase in mregDCs, IFN-primed DCs, CD207 and LTB DC2+DC3s in tumours (not shown). For datasets where healthy tissue, tumour periphery and tumour core had been analysed separately, they confirmed that mregDCs trended to accumulate in the tumour periphery, while proliferating DCs and the DC3/cDC2 ratio trended to accumulate in the tumour core (not shown). Next, they included the predicted mega-cluster from data of Cheng et al., (Cheng et al., 2021) that was mapped using the Azimuth algorithm (Hao et al., 2021) (not shown). This meta-analysis revealed a significant increase of CD207 DC2+DC3s in all cancers included in this study, while IFN-primed DCs, mregDCs, the DC3/cDC2 ratio and proliferating DCs significantly accumulated in most tumours (not shown). Conversely, a significant decrease in cDC1s was also observed. Interestingly, when examining the constitution of proliferating cells using label transfer (Seurat V4), the frequency of CD207 DC2+DC3s among proliferating DCs exhibited a 10.75-fold increase in the tumour relative to healthy tissue (not shown). Moreover, compared to other mega-clusters, CD207 DC2+DC3s and cDC1s comprised a greater percentage of proliferating cells in the tumour than in healthy (not shown).

### Validation of DC Heterogeneity in Non-Small-Cell Lung Carcinoma Patients

Following the identification of markers unravelling antigen presenting DCs' heterogeneity and the characterization of their putative relative changes in cancer, the inventors examined whether these observations could be validated by multiparametric flow cytometry in healthy and tumoural lesions from patients. Among CD45⁺ leukocytes, they first identified the mononuclear phagocytes (MNPs) from normal adjacent and tumour tissue from non-small-cell lung carcinoma (NSCLC) patients (n=8), from which they identified cDC1s, DC2+DC3s, CD123⁺ DCs, and monocyte/macrophages (MoMac) (FIG. 1A and FIG. 5A). MNPs were exported and reanalyzed, revealing all major MNP subsets (FIG. 1B and FIG. 5B). They then focused on specific DC populations, including DC2s and DC3s, where an increase in the percentage of CD14⁺ DC3s was observed in the tumour from 7 of the 8 patients (FIGS. 1C-E). The DC-VERSE revealed CD207 and CD1A were uniquely expressed genes in the CD207 DC2+DC3s, and CITE-seq data also indicated that CD103 was explicitly expressed at the membrane of LTB DC2+DC3s (FIG. 1F). Consequently, they analysed CD1a, CD207 and CD103 at the protein level (FIG. 5D), and while CD103⁺ “LTB” DC2+DC3s were not expanded in tumours (FIGS. 1G-H), they confirmed that CD1a⁺CD207⁺ DC2+DC3s were expanded in the tumour tissue in all patients (FIGS. 1I-J and FIG. 5E). They next looked at CD5 and CD14 expression by CD103⁺ “LTB” DC2+DC3s and CD1a⁺CD207⁺ DC2+DC3s and observed that these two DC populations could both be classified as CD5⁺ DC2 or CD5⁻CD14^{+/-} DC3s. Thus, LTB DC2+DC3s and CD207 DC2+DC3s could rather be two different activation states that both DC2 and DC3s could adopt since they did not constitute a specific subset of either DC2s or DC3s (FIG. 5F).

### Spatial Mapping of CD207 DC2+DC3s in Breast and Lung Tumours

DC heterogeneity meta-analysis revealed that CD207 DC2+DC3s were significantly increased in all examined tumour tissues compared to normal adjacent tissue. Given the expansion of these cells in tumour tissue, the inventors explored where the CD207 DC2+DC3s were located in tumour lesions from three triple-negative breast cancer (TNBC) patients and from two estrogen receptor-positive (ER) patients. To this end, they re-analysed Visium spatial data of breast cancer tumours (Wu et al., 2021), where they observed that contrary to most other myeloid cell subsets [that were often found in immune enriched/tertiary lymphoid structures (TLS) niches], CD207 DC2+DC3s were detected both in ‘invasive cancer+stroma’ and ‘normal+stroma+immune’ niches. They next carried out immuno-histo fluorescence (IHF) analyses on formalin-fixed paraffin-embedded (FFPE) non-small-cell lung carcinoma (NSCLC) tumours obtained from 16 patients (FIGS. 2A-B). Normal adjacent tissue and tumour regions were defined by a pathologist (GG) and are delineated in green and brown, respectively. Quantification of CD207⁺ cells (CD207 DC2+DC3s) confirmed the significantly higher density of CD207⁺ DC2+DC3s in the tumour compared to the normal adjacent lung (FIGS. 2A-B). TLS were detected, and CD207⁺ DC2+DC3s appeared as mostly excluded from these structures (not shown). Within the tumour, while CD3⁺ T-cells (including CD3⁺CD8⁺ T-cells) and CD20⁺ B cells appeared to be accumulating in the tumour stroma, CD207⁺ DC2+DC3s were detected in tumour nests, particularly enriched in glandular structures (not shown). Use of the Halo10 software allowed to perform an unsupervised delineation of the stroma and tumour area only in 13 of the 16 stained cross-sections. Quantitative analyses confirmed the significantly higher density of CD207⁺ DC2+DC3s in the tumour nests as compared to the tumour stroma, while CD3⁺CD8⁺ T-cells were accumulating more in the tumour stroma than the tumour nests (FIGS. 2C-D). Thus, these data not only confirmed the intratumoural accumulation of CD207 DC2+DC3s but also allowed us to observe that these cells were embedded in between tumour cells, where T and B cells were mostly absent.

To corroborate these observations at the single cell resolution, we analysed one lung and one breast cancer patient using the Merfish technology (Merscope, Vizgen). As mentioned above, cell segmentation followed by a sequential dimensionality reduction approach allowed us to define tumour and stroma niches within the two tumours analysed, as well as major immune cell populations (B cells, major T-cell subsets) and most DC subsets and states. This revealed in an unprecedented manner the spatial localisation of all these cells, including the discrete DC populations, whose identification usually requires multiple markers and, to our knowledge, had never been defined at such resolution in human tissue cross-sections. A top DEG of CD207 DC2+DC3s defined in the mDC-VERSE was TNF, an observation that we confirmed here since these cells were the only cells among immune cells in which TNF transcripts were abundantly detected. In the two tumour cross-sections, similarly to most lymphocytes, all DC subsets and states, except for the CD207 DC2+DC3s, were detected in greater abundance within the tumour stroma. CD207 DC2+DC3s were the only DCs detected in greater abundance within the tumour nests (surrounded by tumour cells).

To validate this observation, we carried out 4-color immuno-histofluorescent (IHF) stainings of 16 lung adenocarcinoma patients focusing on CD207⁺ DC2+DC3s. Our IHF data also confirmed a significant intratumoral accumulation of CD207⁺ DC2+DC3s specifically within the tumour glandular areas of patients' tumours (tumour nests), while B and T lymphocytes, including CD8⁺ T-cells, were significantly accumulating in the tumour stroma. Together with the inverse correlation of CD207 DC2+DC3 frequency with that of CD8⁺ T-cells (particularly CD8⁺ T_{RM} cells), this observation allows us to hypothesize that CD207 DC2+DC3s could participate in the regulation of the anti-tumour T-cell cytotoxicity.

CD207 DC2+DC3s are Associated with an Unfavourable Outcome in Cancer

Since cDCs are professional antigen presenting cells that can prime naïve T-cells or reactivate memory T-cells, we explored the potential relationship of CD207 DC2+DC3s with T-cells. To address this, the inventors mapped onto the DC-VERSE (using the Azimuth pipeline) the breast cancer dataset from Bassez et al., where T-cell clonality has been evaluated prior to and after anti-PD-1 therapy (FIG. 3A-C). Contrary to mregDCs, IFN-primed DCs and cDC1s that accumulated in patients with a clonal T-cell expansion, CD207 DC2+DC3s accumulated only in tumours of patients without a clonal T-cell expansion (FIG. 3A). Interestingly, T-cell clonal expansion was only observed in patients with a cDC1 to CD207 DC2+DC3 ratio>1 (both pre- and post-treatment) (FIG. 3B). T-cell clonality quantification requires single-cell TcR-sequencing, which cannot be used routinely due to cost and technical limitations. The CD207 DC2+DC3s to cDC1s ratio could become a mean to predict T-cell clonality within tumours. The inventors next observed that the frequency of CD207 DC2+DC3s tended to negatively correlate with the frequency of CD8⁺ T-cells (FIG. 3C). In addition, CD207 DC2+DC3s inversely correlated with resident memory CD8⁺ T-cells (T_{RMs}) (FIG. 3C) for patients without T-cell clonal expansion. This trend was extended to lung tumours scRNAseq data, where the percentage of CD207 DC2+DC3s trended to be higher in patients with a low frequency of CD8⁺ T_{RMs} (FIG. 4B). Furthermore, CD207 DC2+DC3s strongly correlated with effector memory T-cells that re-express CD45RA (T_{EMRAs}), which are terminally differentiated senescent and hypofunctional cells (Reading et al., 2018) (FIG. 3C). Reanalysing the flow cytometry data, the inventors also observed that the frequencies of CD207 DC2+DC3s significantly negatively correlated with those of CD8⁺ T-cells, but not with that of CD4⁺ T-cells (FIG. 3D and FIG. 4C).

To confirm these observations in other sets of data, the inventors analysed breast cancer (BRCA) and lung adenocarcinoma (LUAD) bulk RNA-seq data obtained from the Cancer Genome Atlas Program (TCGA; FIG. 3E). DC populations signatures were obtained from the DC-VERSE, and other signatures were from the study of Nalio Ramos et al., (Nalio Ramos et al., 2022). In both the BRCA and LUAD cohorts, the CD207 DC2+DC3 signature correlated negatively with T-cell subset signatures (CD8⁺ T-cells, CD8⁺ T_{RMs}, exhausted T-cells and only for LUAD, Tregs) and with the cDC1 signature, while the signatures of the other DC subsets did not show such negative correlation, except for the DC2 signature in the BRCA cohort which also negatively correlated only with the CD8⁺ T_{RM} signature. Also, among the different T-cell subsets, the signature of CD8⁺ T_{RMs} was the most negatively correlated with that of CD207 DC2+DC3s.

Finally, different cohorts of patients were explored. Their tumours were sampled and analyzed by bulk RNAseq before receiving an immune checkpoint blockade treatment (Lánczky and Györffy, 2021), in order to evaluate the prognostic value when using the gene signatures of major DC populations (FIG. 3G).

The following cancer types were thus pooled and analyzed:
Bladder Carcinoma (n=405)
Breast cancer (n=1090)
Cervical squamous cell carcinoma (n=304)
Esophageal Adenocarcinoma (n=80)
Esophageal Squamous Cell Carcinoma (n=81)
Head-neck squamous cell carcinoma (n=500)
Kidney renal clear cell carcinoma (n=530)
Kidney renal papillary cell carcinoma (n=288)
Liver hepatocellular carcinoma (n=371)
Lung adenocarcinoma (n=513)
Lung squamous cell carcinoma (n=501)
Ovarian cancer (n=374)
Pancreatic ductal adenocarcinoma (n=177)
Pheochromocytoma and Paraganglioma (n=178)
Rectum adenocarcinoma (n=165)
Sarcoma (n=259)
Stomach adenocarcinoma (n=375)
Testicular Germ Cell Tumor (n=134)
Thymoma (n=119)
Thyroid carcinoma (n=502)
Uterine corpus endometrial carcinoma (n=543)

While the mean expression of cDC1s (XCR1, CLEC9A) and mregDCs (LAMP3, BIRC3) gene signatures showed a statistically significant increase in overall survival (OS), the mean expression of CD207 DC2+DC3s (CD207, CD1A) gene signature had a statistically significant decrease in OS. There is also a statistical significant increase in OS when looking at individual defining markers for each DC population (XCR1/CADM1/CLEC9A for cDC1, LAMP3/BIRC3/MARCKS for mregDCs, and CD1C, a pan DC2+DC3 gene. Among DC2+DC3, the “CD207” state was specifically of poor prognosis since the expression of CD1C, a pan DC2+DC3 gene, was significantly associated with an increased OS (data not shown). The ratio of cDC1 to CD207 DC2+DC3 transcripts (XCR1/CD207) was also calculated and was very significantly predictive of the survival of patients (FIG. 3G), consistent with the observations made when looking at expansion of T-cell clonality (FIGS. 3A,B). Altogether, this analysis suggests that patients with low CD207 DC2+DC3 and high cDC1 and mregDC tumour infiltrates respond better to ICB treatment and vice versa. The establishment of a score based on the relative expression of cDC1 and CD207 DC2+DC3 transcripts (by RNAseq) and/or proteins (by flow cytometry) could become a prognostic tool to predict response to ICB treatment. Finally, the inventors further explored TCGA datasets from seven cancers (including the BRCA and LUAD cohorts) to evaluate the prognostic value of the CD207 DC2+DC3 signature (FIG. 3F). Patients from these seven cohorts were categorized as positive or negative for the CD207 DC2+DC3 signature. This patient stratification has been based on the mean expression of the CD207 DC2+DC3 signature genes (CD1A, CD207 and HLA-DQB2 transcripts, not shown). When exploring the disease-specific survival (DSS) in these seven TCGA datasets, the frequency of CD207 DC2+DC3 signature-positive patients was consistently increased in cancer-related dead patients (deceased) as compared to alive patients (FIG. 3F).

To confirm these results on other specific cancer types, the impact of the CD207 DC signature on the prediction of response to Immune Checkpoint antibodies inhibitors was studied by using the data displayed in public databases (https://bioinfo.vanderbilt.eu/database/Cancer-Immu and https://bioinfo.life.hust.edu.cn/ICBatlas #!/).

As shown in FIG. 3H, the expression of CD207 CD2+CD3 was strongly enhanced in patients suffering from metastatic Urothelial cancer and melanoma cancer that do not respond to the anti-PD-1 antibody treatment.

Moreover, the expression of CD207 CD2+CD3 was strongly enhanced in patients suffering from NSCLC cancer that do not respond to several treatments (SRP1834555, SRP217040, ERP105482, etc). Also, the expression of CD207 CD2+CD3 was strongly enhanced in patients suffering from a glioblastoma that do not respond to the treatment (SRP155030).

These data could provide potential therapeutic targets in favour of reducing the abundance of CD207 DC2+DC3s. Furthermore, CD207 DC2+DC3s could also serve as a prognostic factor alongside cDC1s as the ratio of these cells predicted whether patients would develop a T-cell clonal expansion.

### BIBLIOGRAPHIC REFERENCES

Bassez, A., Vos, H., Van Dyck, L., Floris, G., Arijs, I., Desmedt, C., Boeckx, B., Vanden Bempt, M., Nevelsteen, I., Lambein, K., et al. (2021). A single-cell map of intratumoural changes during anti-PD1 treatment of patients with breast cancer. Nat Med 27, 820-832.
Becht, E., McInnes, L., Healy, J., Dutertre, C.-A., Kwok, I. W. H., Ng, L. G., Ginhoux, F., and Newell, E. W. (2018). Dimensionality reduction for visualizing single-cell data using UMAP. Nat. Biotechnol.
Bell, D., Chomarat, P., Broyles, D., Netto, G., Harb, G. M., Lebecque, S., Valladeau, J., Davoust, J., Palucka, K. A., and Banchereau, J. (1999). In Breast Carcinoma Tissue, Immature Dendritic Cells Reside within the Tumour, Whereas Mature Dendritic Cells Are Located in Peritumoural Areas. Journal of Experimental Medicine 190, 1417-1426.
Bourdely, P., Anselmi, G., Vaivode, K., Ramos, R. N., Missolo-Koussou, Y., Hidalgo, S., Tosselo, J., Nuñez, N., Richer, W., Vincent-Salomon, A., et al. (2020). Transcriptional and Functional Analysis of CD1c+ Human Dendritic Cells Identifies a CD163+ Subset Priming CD8+CD103+ T Cells. Immunity 53, 335-352.e8.
Brown, C. C., Gudjonson, H., Pritykin, Y., Deep, D., Lavallée, V.-P., Mendoza, A., Fromme, R., Mazutis, L., Ariyan, C., Leslie, C., et al. (2019). Transcriptional Basis of Mouse and Human Dendritic Cell Heterogeneity. Cell 179, 846-863.e24.
Cheng, S., Li, Z., Gao, R., Xing, B., Gao, Y., Yang, Y., Qin, S., Zhang, L., Ouyang, H., Du, P., et al. (2021). A pan-cancer single-cell transcriptional atlas of tumour infiltrating myeloid cells. Cell 184, 792-809.e23.
Cillo, A. R., Kürten, C. H. L., Tabib, T., Qi, Z., Onkar, S., Wang, T., Liu, A., Duvvuri, U., Kim, S., Soose, R. J., et al. (2020). Immune Landscape of Viral- and Carcinogen-Driven Head and Neck Cancer. Immunity 52, 183-199.e9.
Cytlak, U., Resteu, A., Pagan, S., Green, K., Milne, P., Maisuria, S., McDonald, D., Hulme, G., Filby, A., Carpenter, B., et al. (2019). Differential IRF8 Requirement Defines Two Pathways of Dendritic Cell Development in Humans (Rochester, NY: Social Science Research Network).
Di Pilato, M., Kfuri-Rubens, R., Pruessmann, J. N., Ozga, A. J., Messemaker, M., Cadilha, B. L., Sivakumar, R., Cianciaruso, C., Warner, R. D., Marangoni, F., et al. (2021). CXCR6 positions cytotoxic T cells to receive critical survival signals in the tumour microenvironment. Cell 184, 4512-4530.e22.
Duong, E., Fessenden, T. B., Lutz, E., Dinter, T., Yim, L., Blatt, S., Bhutkar, A., Wittrup, K. D., and Spranger, S. (2021). Type I interferon activates MHC class I-dressed CD11b+ conventional dendritic cells to promote protective anti-tumour CD8+ T cell immunity. Immunity https://doi.org/10.1016/j.immuni.2021.10.020.
Dutertre, C.-A., Becht, E., Irac, S. E., Khalilnezhad, A., Narang, V., Khalilnezhad, S., Ng, P. Y., van den Hoogen, L. L., Leong, J. Y., Lee, B., et al. (2019). Single-Cell Analysis of Human Mononuclear Phagocytes Reveals Subset-Defining Markers and Identifies Circulating Inflammatory Dendritic Cells. Immunity 51, 573-589.e8.
Dyduch G. et al.: “CD207+/langerin positive dendritic cells in invasive and in situ cutaneous malignant melanoma”, ADVANCES IN DERMATOLOGY AND ALLERGOLOGY, vol. 3, 9 May 2017
Gerhard, G. M., Bill, R., Messemaker, M., Klein, A. M., and Pittet, M. J. (2020). Tumour-infiltrating dendritic cell states are conserved across solid human cancers. Journal of Experimental Medicine 218.
Ginhoux, F., Guilliams, M., and Merad, M. (2022). Expanding dendritic cell nomenclature in the single-cell era. Nat Rev Immunol 22, 67-68.
Guilliams, M., Ginhoux, F., Jakubzick, C., Naik, S. H., Onai, N., Schraml, B. U., Segura, E., Tussiwand, R., and Yona, S. (2014). Dendritic cells, monocytes and macrophages: a unified nomenclature based on ontogeny. Nat. Rev. Immunol. 14, 571-578.
Guilliams, M., Dutertre, C.-A., Scott, C. L., McGovern, N., Sichien, D., Chakarov, S., Van Gassen, S., Chen, J., Poidinger, M., De Prijck, S., et al. (2016). Unsupervised High-Dimensional Analysis Aligns Dendritic Cells across Tissues and Species. Immunity 45, 669-684.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29.
Kim, N., Kim, H. K., Lee, K., Hong, Y., Cho, J. H., Choi, J. W., Lee, J.-I., Suh, Y.-L., Ku, B. M., Eum, H. H., et al. (2020). Single-cell RNA sequencing demonstrates the molecular and cellular reprogramming of metastatic lung adenocarcinoma. Nature Communications 11, 2285.
Kvedaraite, E., and Ginhoux, F. (2022). Human dendritic cells in cancer. Sci Immunol 7, eabm9409.
Ösz, Á., Lánczky, A., and Györffy, B. (2021). Survival analysis in breast cancer using proteomic data from four independent datasets. Sci Rep 11, 16787. 10.1038/s41598-021-96340-5.
Leader, A. M., Grout, J. A., Maier, B. B., Nabet, B. Y., Park, M. D., Tabachnikova, A., Chang, C., Walker, L., Lansky, A., Le Berichel, J., et al. (2021). Single-cell analysis of human non-small cell lung cancer lesions refines tumour classification and patient stratification. Cancer Cell S1535-6108 (21) 00560-2.
Lee, J., Breton, G., Oliveira, T. Y. K., Zhou, Y. J., Aljoufi, A., Puhr, S., Cameron, M. J., Sékaly, R.-P., Nussenzweig, M. C., and Liu, K. (2015). Restricted dendritic cell and monocyte progenitors in human cord blood and bone marrow. J. Exp. Med. 212, 385-399.
Levine, J. H., Simonds, E. F., Bendall, S. C., Davis, K. L., Amir el, A. D., Tadmor, M. D., Litvin, O., Fienberg, H. G., Jager, A., Zunder, E. R., et al. (2015). Data-Driven Phenotypic Dissection of AML Reveals Progenitor-like Cells that Correlate with Prognosis. Cell 162, 184-197.
Liu, K., Victora, G. D., Schwickert, T. A., Guermonprez, P., Meredith, M. M., Yao, K., Chu, F.-F., Randolph, G. J., Rudensky, A. Y., and Nussenzweig, M. (2009). In vivo analysis of dendritic cell development and homeostasis. Science 324, 392-397.
Mackay, L. K., Minnich, M., Kragten, N. A. M., Liao, Y., Nota, B., Seillet, C., Zaid, A., Man, K., Preston, S., Freestone, D., et al. (2016). Hobit and Blimp1 instruct a universal transcriptional program of tissue residency in lymphocytes. Science 352, 459-463.
Maier, B., Leader, A. M., Chen, S. T., Tung, N., Chang, C., LeBerichel, J., Chudnovskiy, A., Maskey, S., Walker, L., Finnigan, J. P., et al. (2020). A conserved dendritic-cell regulatory program limits antitumour immunity. Nature 580, 257-262.
McInnes, L., Healy, J., and Melville, J. (2018). UMAP: Uniform Manifold Approximation and Projection for Dimension Reduction. ArXiv: 1802.03426 [Cs, Stat].
Mulder, K., Patel, A. A., Kong, W. T., Piot, C., Halitzki, E., Dunsmore, G., Khalilnezhad, S., Irac, S. E., Dubuisson, A., Chevrier, M., et al. (2021). Cross-tissue single-cell landscape of human monocytes and macrophages in health and disease. Immunity 54, 1883-1900.e5.
Naik, S. H., Sathe, P., Park, H.-Y., Metcalf, D., Proietto, A. I., Dakic, A., Carotta, S., O'Keeffe, M., Bahlo, M., Papenfuss, A., et al. (2007). Development of plasmacytoid and conventional dendritic cell subtypes from single precursor cells derived in vitro and in vivo. Nat Immunol 8, 1217-1226.
Nalio Ramos, R., Missolo-Koussou, Y., Gerber-Ferder, Y., Bromley, C. P., Bugatti, M., Núñez, N. G., Tosello Boari, J., Richer, W., Menger, L., Denizeau, J., et al. (2022). Tissue-resident FOLR2+ macrophages associate with CD8+ T cell infiltration in human breast cancer. Cell 185, 1189-1207.e25.
Nussenzweig, M. C., Steinman, R. M., Gutchinov, B., and Cohn, Z. A. (1980). Dendritic cells are accessory cells for the development of anti-trinitrophenyl cytotoxic T lymphocytes. J Exp Med 152, 1070-1084.
Onai, N., Obata-Onai, A., Schmid, M. A., Ohteki, T., Jarrossay, D., and Manz, M. G. (2007). Identification of clonogenic common Flt3+M-CSFR+ plasmacytoid and conventional dendritic cell progenitors in mouse bone marrow. Nat Immunol 8, 1207-1216.
Park, S. L., Gebhardt, T., and Mackay, L. K. (2019). Tissue-Resident Memory T Cells in Cancer Immunosurveillance. Trends in Immunology 40, 735-747.
Qian, J., Olbrecht, S., Boeckx, B., Vos, H., Laoui, D., Etlioglu, E., Wauters, E., Pomella, V., Verbandt, S., Busschaert, P., et al. (2020). A pan-cancer blueprint of the heterogeneous tumour microenvironment revealed by single-cell profiling. Cell Res 30, 745-762.
Reading, J. L., Gálvez-Cancino, F., Swanton, C., Lladser, A., Peggs, K. S., and Quezada, S. A. (2018). The function and dysfunction of memory CD8+ T cells in tumour immunity. Immunological Reviews 283, 194-212.
See, P., Dutertre, C.-A., Chen, J., Günther, P., McGovern, N., Irac, S. E., Gunawan, M., Beyer, M., Händler, K., Duan, K., et al. (2017). Mapping the human DC lineage through the integration of high-dimensional techniques. Science 356.
Steinman, R. M., and Cohn, Z. A. (1973). IDENTIFICATION OF A NOVEL CELL TYPE IN PERIPHERAL LYMPHOID ORGANS OF MICE. The Journal of Experimental Medicine 137, 1142-1162.
Steinman, R. M., and Witmer, M. D. (1978). Lymphoid dendritic cells are potent stimulators of the primary mixed leukocyte reaction in mice. Proc Natl Acad Sci USA 75, 5132-5136.
Steinman, R., Gutchinov, B., Witmer, M., and Nussenzweig, M. (1983). Dendritic cells are the principal stimulators of the primary mixed leukocyte reaction in mice. J Exp Med 157, 613-627.
Stuart, T., Butler, A., Hoffman, P., Hafemeister, C., Papalexi, E., Mauck, W. M., Hao, Y., Stoeckius, M., Smibert, P., and Satija, R. (2019). Comprehensive Integration of Single-Cell Data. Cell 177, 1888-1902.e21.
Tsuji T, Eng K H, Matsuzaki J, Battaglia S, Szender J B, Miliotto A, Gnjatic S, Bshara W, Morrison C D, Lele S, Emerson R O, Wang J, Liu S, Robins H, Lugade A A, Odunsi K. Clonality and antigen-specific responses shape the prognostic effects of tumor-infiltrating T cells in ovarian cancer. Oncotarget. 2020 Jul. 7; 11 (27): 2669-2683.
Valpione S, Mundra P A, Galvani E, Campana L G, Lorigan P, De Rosa F, Gupta A, Weightman J, Mills S, Dhomen N, Marais R. The T cell receptor repertoire of tumor infiltrating T cells is predictive and prognostic for cancer survival. Nat Commun. 2021 Jul. 2; 12 (1): 4098.
Van de Sande, B., Flerin, C., Davie, K., De Waegeneer, M., Hulselmans, G., Aibar, S., Seurinck, R., Saelens, W., Cannoodt, R., Rouchon, Q., et al. (2020). A scalable SCENIC workflow for single-cell gene regulatory network analysis. Nature Protocols 15, 2247-2276.
Villani, A.-C., Satija, R., Reynolds, G., Sarkizova, S., Shekhar, K., Fletcher, J., Griesbeck, M., Butler, A., Zheng, S., Lazo, S., et al. (2017). Single-cell RNA-seq reveals new types of human blood dendritic cells, monocytes, and progenitors. Science 356.
Wu, S. Z., Al-Eryani, G., Roden, D. L., Junankar, S., Harvey, K., Andersson, A., Thennavan, A., Wang, C., Torpy, J. R., Bartonicek, N., et al. (2021). A single-cell and spatially resolved atlas of human breast cancers. Nat Genet 53, 1334-1347.
Zhang, Q., He, Y., Luo, N., Patel, S. J., Han, Y., Gao, R., Modak, M., Carotta, S., Haslinger, C., Kind, D., et al. (2019). Landscape and Dynamics of Single Immune Cells in Hepatocellular Carcinoma. Cell 179, 829-845.e20.
Zhang, Y., Chen, H., Mo, H., Hu, X., Gao, R., Zhao, Y., Liu, B., Niu, L., Sun, X., Yu, X., et al. (2021). Single-cell analyses reveal key immune cell subsets associated with response to PD-L1 blockade in triple-negative breast cancer. Cancer Cell 39, 1578-1593.e8.
Zheng, C., Zheng, L., Yoo, J.-K., Guo, H., Zhang, Y., Guo, X., Kang, B., Hu, R., Huang, J. Y., Zhang, Q., et al. (2017). Landscape of Infiltrating T Cells in Liver Cancer Revealed by Single-Cell Sequencing. Cell 169, 1342-1356.e16.
Ziegler-Heitbrock, L., Ancuta, P., Crowe, S., Dalod, M., Grau, V., Hart, D. N., Leenen, P. J. M., Liu, Y.-J., MacPherson, G., Randolph, G. J., et al. (2010). Nomenclature of monocytes and dendritic cells in blood. Blood 116, e74-80.
Zilionis, R., Engblom, C., Pfirschke, C., Savova, V., Zemmour, D., Saatcioglu, H. D., Krishnan, I., Maroni, G., Meyerovitz, C. V., Kerwin, C. M., et al. (2019). Single-Cell Transcriptomics of Human and Mouse Lung Cancers Reveals Conserved Myeloid Populations across Individuals and Species. Immunity 50, 1317-1334.e10.

1.-20. (canceled)

21. A method to prognose the evolution of a solid cancer in a subject in need thereof, said method comprising the steps of:
a) detecting and quantifying quantity, density or frequency of CD207⁺ conventional dendritic cells of type 2 (DC2+DC3) cell population in a tumor sample of said subject,
b) optionally, detecting and quantifying quantity, density or frequency of conventional dendritic cells of type 1 (cDC1) cells in the same tumor sample,
c) comparing the value of step a) to a reference value or comparing the value of step a) to the value obtained in step b), and
d) prognosing that the subject has a bad prognosis if the quantity, density or frequency of CD207⁺ DC2+DC3 cell population is higher than said reference value or if the quantity, density or frequency of CD207⁺ DC2+DC3 cell population is higher than the quantity, density or frequency of cDC1 cells.

22. The method of claim 21, wherein step a) comprises assessing CD207⁺ DC2+DC3 density by immunohistochemistry on a sample of the tumor.

23. The method of claim 21, wherein step a) comprises assessing the frequency of CD207⁺ DC2+DC3 cells among CD45⁺ leukocytes on a sample of the tumor.

24. The method of claim 23, wherein said step a) is performed by flow cytometry.

25. The method of claim 21, wherein step a) is performed by measuring the quantity of CD1A and CD207 mRNAs in a RNA sample of the tumor core.

26. The method of claim 21, wherein the tumor sample is a sample of the tumor core and if the quantity, density or frequency of CD207⁺ DC2+DC3 is increased in a sample of the tumor core, then the prognosis of the subject is bad.

27. The method of claim 21, wherein said solid tumor is a carcinoma or a sarcoma selected from lung cancer, malignant mesothelioma, bladder cancer, kidney cancer, testicular cancer, breast cancer, cancer of the upper aero-digestive tract, liver cancer, pancreas cancer, stomach cancer; colon cancer and ovarian cancer.

28. The method of claim 21, wherein said reference value has been obtained from normal adjacent tissue of the same subject.

29. The method of claim 21, wherein steps a) and b) are performed by exclusion gating flow cytometry, by immunohistochemistry, or by RNAseq.

30. The method of claim 21, wherein it is used to evaluate the response of a patient to an anti-cancer treatment.

31. The method of claim 30, wherein said anti-cancer treatment is an immune checkpoint blocker selected from anti-CTLA4 (ipilimumab and Tremelimumab), anti-PD-1 (Nivolumab and Pembrolizumab), anti-PD-L1 (Atezolizumab, Durvalumab, and Avelumab), anti-PD-L2 and anti-Tim3, and combinations thereof.

32. A method for treating a solid cancer in a subject in need thereof, comprising administering a cytotoxic antibody that specifically binds to CD207⁺ DC2+DC3 cells.

33. The method of to claim 32, wherein the cytotoxic antibody that specifically binds to CD207⁺ DC2+DC3 cells is administered intratumorally.

34. The method of claim 32, wherein the cytotoxic antibody that specifically binds to CD207⁺ DC2+DC3 cells is a humanized antibody carrying a cytotoxic drug, or a humanized antibody modified so as to mediate complement-dependent cytotoxicity (CDC) or Antibody-Dependent Cellular Cytotoxicity (ADCC).

35. The method of claim 21, wherein step a) is performed by flow cytometry, by measuring the expression of CD1a and CD207 biomarkers at the surface of the CD45⁺ leukocytes.

36. The method of claim 21, wherein the tumor sample is a sample of a tumor core comprising a glandular structure of a tumor nest, and if quantity, density or frequency of CD207⁺ DC2+DC3 is increased in a glandular structure of a tumor nest, as compared to the reference value, then the prognosis of the subject is bad.

37. The method of claim 21, wherein said solid tumor is non small-cell lung carcinoma (NSCLC) or triple-negative breast cancer (TNBC).
